SEQUENCE LISTING (24 sequences)

SEQ ID NO: 1 - protein, 292 aa, homo sapiens
MAFMKKYLLPILGLFMAYYYYSANEEFRPEMLQGKKVIVTGASKGIGREMAYHLAKMGAHVVVTARSKETLQKVVSHCLELGAASAHYIAGTMEDMTFAEQFVAQAGKLMGGLDMLILNHITNTSLNLFHDDIHHVRKSMEVNFLSYVVLTVAALPMLKQSNGSIVVVSSLAGKVAYPMVAAYSASKFALDGFFSSIRKEYSVSRVNVSITLCVLGLIDTETAMKAVSGIVHMQAAPKEECALEIIKGGALRQEEVYYDSSLWTTLLIRNPCRKILEFLYSTSYNMDRFINK

SEQ ID NO: 2 - protein, 416 aa, homo sapiens
MPRQLSAAAALFASLAVILHDGSQMRAKAFPETRDYSQPTAAATVQDIKKPVQQPAKQAPHQTLAARFMDGHITFQTAATVKIPTTTPATTKNTATTSPITYTLVTTQATPNNSHTAPPVTEVTVGPSLAPYSLPPTITPPAHTTGTSSSTVSHTTGNTTQPSNQTTLPATLSIALHKSTTGQKPVQPTHAPGTTAAAHNTTRTAAPASTVPGPTLAPQPSSVKTGIYQVLNGSRLCIKAEMGIQLIVQDKESVFSPRRYFNIDPNATQASGNCGTRKSNLLLNFQGGFVNLTFTKDEESYYISEVGAYLTVSDPETIYQGIKHAVVMFQTAVGHSFKCVSEQSLQLSAHLQVKTTDVQLQAFDFEDDHFGNVDECSSDYTIVLPVIGAIVVGLCLMGMGVYKIRLRCQSSGYQRI

SEQ ID NO: 3 - protein, 525 aa, homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 4 - protein, 94 aa, homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 5 - protein, 99 aa, homo sapiens
MKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

SEQ ID NO: 6 - protein, 530 aa, homo sapiens
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQAATKQSNASSDVEVEEKETSVSKEDTDHEEKASNEDVTKAVDITTPKAARRGRKRKAEKQVETEEAGVVTTATASVNLKVSPKRGRPAATEVKIPKPRGRPKMVKQPCPSESDIITEEDKSKKKGQEEKQPKKQPKKDEEGQKEEDKPRKEPDKKEGKKEVESKRKNLAKTGVTSTSDSEEEGDDQEGEKKRKGGRNFQTAHRRNMLKGQHEKEAADRKRKQEEQMETEQQNKDEGKKPEVKKVEKKRETSMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSLAEQRQHEEANKTKDQGKKGPNKKLEKEQTGSKTLNGGSDAQDGNQPQHNGESNEDSKDNHEASTKKKPSSEERETEISLKDSTLDN

SEQ ID NO: 7 - protein, 775 aa, homo sapiens
MASRAVVRARRCPQCPQVRAAAAAPAWAALPLSRSLPPCSNSSSFSMPLFLLLLLVLLLLLEDAGAQQGDGCGHTVLGPESGTLTSINYPQTYPNSTVCEWEIRVKMGERVRIKFGDFDIEDSDSCHFNYLRIYNGIGVSRTEIGKYCGLGLQMNHSIESKGNEITLLFMSGIHVSGRGFLASYSVIDKQDLITCLDTASNFLEPEFSKYCPAGCLLPFAEISGTIPHGYRDSSPLCMAGVHAGVVSNTLGGQISVVISKGIPYYESSLANNVTSVVGHLSTSLFTFKTSGCYGTLGMESGVIADPQITASSVLEWTDHTGQENSWKPKKARLKKPGPPWAAFATDEYQWLQIDLNKEKKITGIITTGSTMVEHNYYVSAYRILYSDDGQKWTVYREPGVEQDKIFQGNKDYHQDVRNNFLPPIIARFIRVNPTQWQQKIAMKMELLGCQFIPKGRPPKLTQPPPPRNSNDLKNTTAPPKIAKGRAPKFTQPLQPRSSNEFPAQTEQTTASPDIRNTTVTPNVTKDVALAAVLVPVLVMVLTTLILILVCAWHWRNRKKKTEGTYDLPYWDRAGWWKGMKQFLPAKAVDHEETPVRYSSSEVNHLSPREVTTVLQADSAEYAQPLVGGIVGTLHQRSTFKPEEGKEAGYADLDPYNSPGQEVYHAYAEPLPITGPEYATPIIMDMSGHPTTSVGQPSTSTFKATGNQPPPLVGTYNTLLSRTDSCSSAQAQYDTPKAGKPGLPAPDELVYQVPQSTQEVSGAGRDGECDVFKEIL

SEQ ID NO: 8 - protein, 247 aa, homo sapiens
MEYHPDLENLDEDGYTQLHFDSQSNTRIAVVSEKGSCAASPPWRLIAVILGILCLVILVIAVVLGTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 9 - protein, 448 aa, homo sapiens
MIPTFTALLCLGLSLGPRTHMQAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWEVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 10 - protein, 252 aa, homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 11 - protein, 99 aa, homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 12 - protein, 541 aa, homo sapiens
MNCRELPLTLWVLISVSTAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 13 - protein, 249 aa, homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 14 - protein, 252 aa, homo sapiens
MGVLLTQRTLLSLVLALLFPSMASMAAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSRRHSPHQALRKGVRRTRPSRKGKRLMTRGQLPR

SEQ ID NO: 15 - protein, 150 aa, homo sapiens
MNLWLLACLVAGFLGAWAPAVHTQGVFEDCCLAYHYPIGWAVLRRAWTYRIQEVSGSCNLPAAIFYLPKRHRKVCGNPKSREVQRAMKLLDARNKVFAKLHHNTQTFQAGPHAVKKLSSGNSKLSSSKFSNPISSSKRNVSLLISANSGL

SEQ ID NO: 16 - protein, 178 aa, homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 17 - protein, 96 aa, homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 18 - protein, 247 aa, homo sapiens
MLQNSAVLLVLVISASATHEAEQNDSVSPRKSRVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA

SEQ ID NO: 19 - protein, 292 aa, homo sapiens
MASSMRSLFSDHGKYVESFRRFLNHSTEHQCMQEFMDKKLPGIIGRIGDTKSEIKILSIGGGAGEIDLQILSKVQAQYPGVCINNEVVEPSAEQIAKYKELVAKTSNLENVKFAWHKETSSEYQSRMLEKKELQKWDFIHMIQMLYYVKDIPATLKFFHSLLGTNAKMLIIVVSGSSGWDKLWKKYGSRFPQDDLCQYITSDDLTQMLDNLGLKYECYDLLSTMDISDCFIDGNENGDLLWDFLTETCNFNATAPPDLRAELGKDLQEPEFSAKKEGKVLFNNTLSFIVIEA

SEQ ID NO: 20 - protein, 199 aa, homo sapiens
MSSGNAKIGHPAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYFSKQK

SEQ ID NO: 21 - protein, 359 aa, homo sapiens
MAEPFLSEYQHQPQTSNCTGAAAVQEELNPERPPGAEERVPEEDSRWQSRAFPQLGGRPGPEGEGSLESQPPPLQTQACPESSCLREGEKGQNGDDSSAGGDFPPPAEVEPTPEAELLAQPCHDSEASKLGAPAAGGEEEWGQQQRQLGKKKHRRRPSKKKRHWKPYYKLTWEEKKKFDEKQSLRASRIRAEMFAKGQPVAPYNTTQFLMDDHDQEEPDLKTGLYSKRAAAKSDDTSDDDFMEEGGEEDGGSDGMGGDGSEFLQRDFSETYERYHTESLQNMSKQELIKEYLELEKCLSRMEDENNRLRLESKRLGGDDARVRELELELDRLRAENLQLLTENELHRQQERAPLSKFGD

SEQ ID NO: 22 - protein, 401 aa, homo sapiens
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 23 - protein, 250 aa, homo sapiens
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKF

SEQ ID NO: 24 - protein, 202 aa, Homo sapiens
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

(19) United States
(12) Patent Application Publication
(10) Pub. No.: US 2024/0103001 A1
(43) Pub. Date: Mar. 28, 2024

(54) MARKERS FOR DIAGNOSING INFECTIONS

(71) Applicant: MeMed Diagnostics Ltd., Tirat HaCarmel (IL)

(72) Inventors: Michal ROSENBERG, Haifa (IL); Oded SHAHAM, Haifa (IL); Einav SIMON, Atlit (IL); Roy NAVON, Tel-Aviv (IL); Alon ANGEL, Haifa (IL); Niv Steven MASTBOIM, Haifa (IL); Eran EDEN, Haifa (IL); Kfir OVED, Hof HaCarmel (IL)

(73) Assignee: MeMed Diagnostics Ltd., Tirat HaCarmel (IL)

(21) Appl. No.: 18/272,838

(22) PCT Filed: Jan. 18, 2022
(86) PCT No.: PCT/IL2022/050076

§ 371 Date: Jul. 18, 2023

Related U.S. Application Data
(60) Provisional application No. 63/138,530, filed on Jan. 18, 2021.
(60) Provisional application No. 63/172,135, filed on Apr. 8, 2021.

(51) Int. Cl. G01N 33/569; G01N 33/68

(52) U.S. Cl. CPC G01N 33/56983; G01N 33/56911; G01N 33/6893; G01N 2800/60

(57) ABSTRACT

A method of classifying an infectious disease in a subject are disclosed.

The method comprises:
(a) measuring the amount of at least one protein set forth in Table 4 and at least one protein set forth in Table 6 in a sample derived from the subject; and
(b) ruling in or ruling out a viral disease based on the amount of the at least one protein set forth in Table 4 and determining the severity of the viral disease based on the amount of the at least one protein set forth in Table 6.

### RELATED APPLICATIONS

This application claims the benefit of priority of U.S. Provisional Applications 63/138,530 filed 18 Jan. 2021 and 63/172,135 filed 8 Apr. 2021, the contents of which are incorporated herein by reference in their entirety.

### SEQUENCE LISTING STATEMENT

The ASCII file, entitled 90424SequenceListing.txt, created on 18 Jan. 2022, comprising 65,536 bytes, submitted concurrently with the filing of this application is incorporated herein by reference.

### FIELD AND BACKGROUND OF THE INVENTION

The present invention, in some embodiments thereof, relates to the identification of signatures and determinants associated with bacterial and viral infections.

Disease assessment is one of the most important tasks in management of infectious disease patients. Complement to determining infection etiology, predicting patient prognosis may affect various aspects of patient management including treatment, diagnostic tests (e.g., microbiology, blood chemistry, radiology etc.), and admission. Timely identification of patients with higher chance for poor prognosis may result in more aggressive patient management procedures including for example, intensive care unit (ICU) admission, advanced therapeutics, invasive diagnostics or surgical intervention, which could reduce complications and mortality.

Additional background art includes WO 2013/117746, WO 2016/024278, WO2018/060998 and WO2018/060999.

### SUMMARY OF THE INVENTION

According to an aspect of the present invention there is provided a method of classifying an infectious disease in a subject comprising:
(a) measuring the amount of at least one protein set forth in Table 4 and at least one protein set forth in Table 6 in a sample derived from the subject; and
(b) ruling in or ruling out a viral disease based on the amount of the at least one protein set forth in Table 4 and determining the severity of the viral disease based on the amount of the at least one protein set forth in Table 6, thereby classifying the infectious disease.

According to an aspect of the present invention there is provided a method of classifying an infectious disease in a subject comprising:
(a) measuring the amount of at least one protein set forth in Table 3 and at least one protein set forth in Table 6 in a sample derived from the subject; and
(b) ruling in or ruling out a bacterial disease based on the amount of the at least one protein set forth in Table 3 and determining the severity of the bacterial disease based on the amount of the at least one protein set forth in Table 6, thereby classifying the infectious disease.

According to an aspect of the present invention there is provided a method of classifying an infectious disease in a subject comprising:
(a) measuring the amount of at least one protein set forth in Table 3, at least one protein set forth in Table 4 and at least one protein set forth in Table 6 in a sample derived from the subject; and
(b) ruling in a bacterial or viral disease based on the amount of the at least one protein set forth in Table 3 or 4 and determining the severity of the infectious disease based on the amount of the at least one protein set forth in Table 6, thereby classifying the infectious disease.

According to an aspect of the present invention there is provided a method of distinguishing between a bacterial and viral infection in a subject showing signs of a severe infection comprising:
(a) measuring the amounts of TRAIL, CRP, IP10 and at least one additional protein selected from the group consisting of LIF, CCL20 and FGF-23; and
(b) ruling in a bacterial or viral disease based on the amounts.

According to an aspect of the present invention there is provided a method of determining the severity of a viral disease in a subject comprising:
(a) measuring the amounts of TRAIL, CRP, IP10 and at least one additional protein selected from the group consisting of KRT19 and MCP-3; and
(b) determining the severity of the viral disease based on the amounts.

According to an aspect of the present invention there is provided a method of classifying an infectious disease in a subject comprising:
(a) measuring the amount of at least one RNA set forth in Tables 8 or 9 and at least one RNA set forth in Tables 10 or 11 in a sample derived from the subject; and
(b) distinguishing between a viral and bacterial disease based on the amount of the at least one RNA set forth in Tables 10 or 11 and determining the severity of the infectious disease based on the amount of the at least one RNA set forth in Tables 8 or 9, thereby classifying the infectious disease.

According to an aspect of the present invention there is provided a method of classifying an infectious disease in a subject comprising:
(a) measuring the amount of at least one RNA set forth in Tables 8 or 9, at least one RNA set forth in Table 10 and at least one RNA set forth in Table 11 in a sample derived from the subject; and
(b) ruling in a bacterial or viral disease based on the amount of the at least one RNA set forth in Table 10 and the at least one RNA set forth in Table 11 and determining the severity of the infectious disease based on the amount of the at least one RNA set forth in Tables 8 or 9, thereby classifying the infectious disease.

According to an aspect of the present invention there is provided a kit for diagnosing an infection type comprising:
(i) a detection reagent which specifically detects at least one protein set forth in Table 4; and
(ii) a detection reagent which specifically detects at least one protein set forth in Table 6.

According to an aspect of the present invention there is provided a method of treating a subject having an infectious disease comprising:
(a) classifying the infection type according to the methods described herein; and
(b) treating the subject according to the classification of the infection, wherein when a severe viral infection is ruled in, at least one of the following treatments is used: treatment with an agent selected from the group consisting of Molnupiravir, Paxlovid and Remdesivir; hospitalization; placement in intensive care; mechanical ventilation; and/or treatment of last resort.

According to an aspect of the present invention there is provided a kit for diagnosing an infection type comprising:
(i) a detection reagent which specifically detects at least one RNA set forth in Tables 8 or 9; and
(ii) a detection reagent which specifically detects at least one RNA set forth in Tables or 11.

4. The method of any one of claims 1-3, wherein the classifying is not based on the amount of a protein that is differentially expressed in both (a) bacterial and viral infections; and (b) in severe and non-severe infections.

According to embodiments of the invention, the at least one protein set forth in Table 4 is selected from the group consisting of LAMP3, LAG3, CXCL11 and MCP-2.

According to embodiments of the invention, the at least one protein set forth in Table 3 is OSM or CCL25.

According to embodiments of the invention, the at least one protein set forth in Table 6 is selected from the group consisting of FGF-23, IL10, CCL20, IL8, STC1, HNMT, AREG, OPG, DCBLD2, PRDX1, PSIP1 and HEXIM1.

According to embodiments of the invention, the at least one protein in Table 4 comprises at least two proteins set forth in Table 4.

According to embodiments of the invention, the at least one protein in Table 4 comprises at least two proteins set forth in Table 4 and the at least one protein in Table 3 comprises at least two proteins set forth in Table 3.

According to embodiments of the invention, the at least one protein in Table 3 comprises at least two proteins set forth in Table 3.

According to embodiments of the invention, the at least one protein in Table 6, comprises at least two proteins set forth in Table 6.

According to embodiments of the invention, the classifying is not based on the amount of a RNA that is differentially expressed in both (a) bacterial and viral infections; and (b) in severe and non-severe infections.

According to embodiments of the invention, the subject shows symptoms of an infectious disease.

According to embodiments of the invention, the subject does not show symptoms of an infectious disease.

According to embodiments of the invention, the subject does not have a chronic non-infectious disease.

According to embodiments of the invention, the sample is whole blood or a fraction thereof.

According to embodiments of the invention, the fraction comprises cells selected from the group consisting of lymphocytes, monocytes and granulocytes.

According to embodiments of the invention, the fraction comprises serum or plasma.

According to embodiments of the invention, the level of no more than 10 proteins is used to classify the infection.

According to embodiments of the invention, no more than 5 proteins are measured to determine the infection type.

According to embodiments of the invention, the kit further comprises a detection reagent which specifically detects at least one protein set forth in Table 3.

According to embodiments of the invention, the kit is devoid of antibodies that specifically detect a protein that is differentially expressed in both (a) bacterial and viral infections; and (b) in severe and non-severe infections.

According to embodiments of the invention, the detection reagent comprises an antibody.

According to embodiments of the invention, the antibody is attached to a detectable moiety.

According to embodiments of the invention, the antibody is a monoclonal antibody.

According to embodiments of the invention, the antibody is attached to a solid support.

According to embodiments of the invention, the kit comprises detection reagents that specifically detect no more than 10 protein markers.

According to embodiments of the invention, the kit comprises detection reagents that specifically detect no more than 6 protein markers.

According to embodiments of the invention, the at least one protein set forth in Table 4 is selected from the group consisting of LAMP3, LAG3, CXCL11 and MCP-2.

According to embodiments of the invention, the at least one protein set forth in Table 6 is selected from the group consisting of FGF-23, IL10, CCL20, IL8, STC1, HNMT, AREG, OPG, DCBLD2, PRDX1, PSIP1 and HEXIM1.

According to embodiments of the invention, the at least one protein set forth in Table 3 is OSM or CCL25.

According to embodiments of the invention, the detection agent comprises a polynulcleotide capable of specifically hybridizing to the at least one RNA set forth in Tables 8 or 9 and a polynulcleotide capable of specifically hybridizing to the at least one RNA set forth in Tables 10 or 11.

According to embodiments of the invention, the detection agent comprises a detectable moiety.

According to embodiments of the invention, the subject shows symptoms of an infectious disease.

According to embodiments of the invention, the symptoms comprise fever.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the Drawings:

FIG. 1 is a Venn diagram illustrating the unique and overlapping biomarkers for severity, bacterial and viral infections.

### DESCRIPTION OF SPECIFIC EMBODIMENTS OF THE INVENTION

The present invention, in some embodiments thereof, relates to the identification of signatures and determinants associated with bacterial and viral infections.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Differentiating between bacterial and viral infections is a daily clinical challenge. Recent publications have shown that the host response to severe infection display an inflammatory ‘bacterial’ pattern, regardless of the underlying infectious etiology, even at the case of an underlying viral infection (Tang, B. M., Nature Communications 10, 3422. doi:10.1038/s41467-019-11249-y; Dunning, J., et al Nature Immunology 19(6):625-635. doi:10.1038/s41590-018-0111-5).

The present inventors have surprisingly uncovered a unique set of immune proteins, found to be specific markers for viral or bacterial infections, which are unaffected by disease severity. Conversely, a set of protein markers have been identified whose expression correlate with disease severity, and are unaffected by the underlying etiology.

In parallel, the present inventors have also uncovered a unique set of immune RNAs, which are specific markers for viral or bacterial infections, which are unaffected by disease severity and another set of RNA markers whose expression correlate with disease severity regardless of the underlying etiology.

The present inventors propose diagnosing subjects and making appropriate treatment decisions based on a combination of such markers.

Thus, according to a first aspect of the present invention, there is provided a method of classifying an infectious disease in a subject comprising:
(a) measuring the amount of at least one protein set forth in Table 4 and at least one protein set forth in Table 6 in a sample derived from the subject; and
(b) ruling in or ruling out a viral disease based on the amount of the at least one protein set forth in Table 4 and determining the severity of the viral disease based on the amount of the at least one protein set forth in Table 6, thereby classifying the infectious disease.

According to another aspect of the present invention there is provided a method of classifying an infectious disease in a subject comprising:
(a) measuring the amount of at least one protein set forth in Table 3 and at least one protein set forth in Table 6 in a sample derived from the subject; and
(b) ruling in or ruling out a bacterial disease based on the amount of the at least one protein set forth in Table 3 and determining the severity of the bacterial disease based on the amount of the at least one protein set forth in Table 6, thereby classifying the infectious disease.

According to still another aspect of the present invention, there is provided a method of classifying an infectious disease in a subject comprising:
(a) measuring the amount of at least one protein set forth in Table 3, at least one protein set forth in Table 4 and at least one protein set forth in Table 6 in a sample derived from the subject; and
(b) ruling in a bacterial or viral disease based on the amount of the at least one protein set forth in Table 3 or 4 and determining the severity of the infectious disease based on the amount of the at least one protein set forth in Table 6, thereby classifying the infectious disease.

A “subject” in the context of the present invention may be a mammal (e.g. human, dog, cat, horse, cow, sheep, pig or goat). According to another embodiment, the subject is a bird (e.g. chicken, turkey, duck or goose). According to a particular embodiment, the subject is a human. The subject may be male or female. The subject may be an adult (e.g. older than 18, 21, or 22 years or a child (e.g. younger than 18, 21 or 22 years). In another embodiment, the subject is an adolescent (between 12 and 21 years), an infant (29 days to less than 2 years of age) or a neonate (birth through the first 28 days of life).

The subject of this aspect of the present invention may have symptoms of an infection.

Exemplary symptoms include, but are not limited to fever, headache, cough, runny nose, chills, muscle aches, loss of taste and/or loss of smell.

According to a particular embodiment, measuring the determinants (e.g. proteins or RNAs) described herein above is carried out no more than 24 hours following the start of symptoms, no more than 36 hours following the start of symptoms, no more than 48 hours following the start of symptoms, no more than 72 hours following the start of symptoms, no more than 96 hours following the start of symptoms, or no more than 1 week following the start of symptoms.

According to another embodiment, the subject is asymptomatic.

It will be appreciated, whether symptomatic or asymptomatic, the subject may or may not be contagious.

In one embodiment, the subject does not have a chronic non-infectious disease such as cancer, a chronic immune disorder or a chronic inflammatory disorder.

In one embodiment, the subject is hospitalized.

In another embodiment, the subject is non-hospitalized.

For any of the aspects disclosed herein, the term “measuring” or “measurement,” or alternatively “detecting” or “detection,” means assessing the presence, absence, quantity or amount (which can be an effective amount) of the determinant within a clinical or subject-derived sample, including the derivation of qualitative or quantitative concentration levels of such determinants.

Methods of measuring the level of protein determinants are well known in the art and include, e.g., immunoassays based on antibodies to proteins, aptamers or molecular imprints.

Protein determinants can be detected in any suitable manner, but are typically detected by contacting a sample from the subject with an antibody, which binds the protein determinant and then detecting the presence or absence of a reaction product. The antibody may be monoclonal, polyclonal, chimeric, or a fragment of the foregoing, and the step of detecting the reaction product may be carried out with any suitable immunoassay.

In one embodiment, the antibody which specifically binds the determinant is attached (either directly or indirectly) to a signal producing label, including but not limited to a radioactive label, an enzymatic label, a hapten, a reporter dye or a fluorescent label.

Immunoassays carried out in accordance with some embodiments of the present invention may be homogeneous assays or heterogeneous assays. In a homogeneous assay the immunological reaction usually involves the specific antibody (e.g., anti-determinant antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels, which may be employed, include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate, pipette tip or slide, and contacted with the specimen suspected of containing the antigen in a liquid phase.

The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunofluorescence methods, immunoprecipitation, chemiluminescence methods, electrochemiluminescence (ECL) or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al., titled “Methods for Modulating Ligand-Receptor Interactions and their Application,” U.S. Pat. No. 4,659,678 to Forrest et al., titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.” The determinant can also be detected with antibodies using flow cytometry. Those skilled in the art will be familiar with flow cytometric techniques which may be useful in carrying out the methods disclosed herein (Shapiro 2005). These include, without limitation, Cytokine Bead Array (Becton Dickinson) and Luminex technology.

Antibodies can be conjugated to a solid support suitable for a diagnostic assay (e.g., beads such as magnetic beads, protein A or protein G agarose, microspheres, plates, slides, pipette tip or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

In particular embodiments, the antibodies of the present invention are monoclonal antibodies.

Suitable sources for antibodies for the detection of determinants include commercially available sources such as, for example, Abazyme, Abnova, AssayPro, Affinity Biologicals, AntibodyShop, Aviva bioscience, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immunostar, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysiences, Inc., Promega Corporation, Proteogenix, Protos Immunoresearch, QED Biosciences, Inc., R&D Systems, Repligen, Research Diagnostics, Robo screen, Santa Cruz Biotechnology, Seikagaku America, Serological Corporation, Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. However, the skilled artisan can routinely make antibodies, against any of the polypeptide determinants described herein.

The presence of a label can be detected by inspection, or a detector which monitors a particular probe or probe combination is used to detect the detection reagent label. Typical detectors include spectrophotometers, phototubes and photodiodes, microscopes, scintillation counters, cameras, film and the like, as well as combinations thereof. Those skilled in the art will be familiar with numerous suitable detectors that widely available from a variety of commercial sources and may be useful for carrying out the method disclosed herein. Commonly, an optical image of a substrate comprising bound labeling moieties is digitized for subsequent computer analysis. See generally The Immunoassay Handbook [The Immunoassay Handbook. Third Edition. 2005].

Examples of antibodies for measuring OSM include without limitation: Rabbit Anti-Human Oncostatin M/OSM Polyclonal Antibody (ab198830) (Abcam), Mouse Anti-Human Oncostatin M/OSM Monoclonal Antibody (R&D Systems), Rabbit Anti-Human Oncostatin M/OSM Polyclonal Antibody (Boster Bio), Mouse Anti-Human Oncostatin M/OSM Monoclonal Antibody (Novus Biologicals).

Examples of antibodies for measuring CCL25 include without limitation: Mouse Anti-Human CCL25/TECK Monoclonal Antibody (MAB3341) (R&D Systems), Rabbit Anti-Human CCL25/TECK Polyclonal Antibody (25285-1-AP) (ThermoFisher), Goat Anti-Human CCL25/TECK Polyclonal Antibody (PA5-47479) (ThermoFisher).

Examples of antibodies for measuring TWEAK include without limitation: Rabbit Anti-Human TWEAK Polyclonal Antibody (ab37170) (Abcam), Goat Anti-Human TWEAK Polyclonal Antibody (Invitrogen), Goat Anti-Human TWEAK/TNFSF12 Polyclonal Antibody (R&D Systems), Rabbit Anti-Human TWEAK/TNFSF12 Polyclonal Antibody (Novus Biologicals), Mouse Anti-Human TWEAK/TNFSF12 Monoclonal Antibody (LifeSpan BioSciences).

Examples of antibodies for measuring HSD11B1 include without limitation: Rabbit Anti-Human HSD11B1 Polyclonal Antibody (ab39364) (Abcam), Rabbit Anti-Human HSD11B1 Polyclonal Antibody (Invitrogen), Rabbit Anti-Human HSD11B1 Polyclonal Antibody (Boster Bio), Rabbit Anti-Human HSD11B1 Polyclonal Antibody (Abbexa).

Examples of antibodies for measuring LAMP3 include without limitation: Rabbit Anti-Human LAMP3 Polyclonal Antibody (Invitrogen), Rabbit Anti-Human LAMP3 Polyclonal Antibody (ab111090) (Abcam), Anti-CD63 (LAMP3) Antibody, clone ME491 (Merck), Rabbit Anti-Human LAMP3/CD208 Polyclonal Antibody (LifeSpan BioSciences).

Examples of antibodies for measuring HGF include without limitation: Rabbit Anti-Human HGF Polyclonal Antibody (ab24865) (Abcam), Mouse Anti-Human HGF Monoclonal Antibody (R&D Systems), Mouse Anti-Human HGF Monoclonal Antibody (Invitrogen), Rabbit Anti-Human HGF Polyclonal Antibody (GeneTex).

Examples of antibodies for measuring TREM1 include without limitation: Mouse Anti-Human TREM-1 Monoclonal Antibody (R&D Systems), Rabbit Anti-Human TREM1 Polyclonal Antibody (Merck), Rabbit Anti-Human TREM1 Polyclonal Antibody (Invitrogen), Mouse Anti-Human CD354 (TREM-1) Monoclonal Antibody (BioLegend).

Examples of antibodies for measuring CXCL11 include without limitation: Rat Anti-Mouse CXCL11/I-TAC Monoclonal Antibody (R&D Systems), Goat Anti-Human CXCL11/I-TAC Polyclonal Antibody (R&D Systems), Rabbit Anti-Human CXCL11 Polyclonal Antibody (ab9955) (Abcam), Mouse Anti-Human CXCL11 Monoclonal Antibody (Invitrogen).

Examples of antibodies for measuring LAG3 include without limitation: Goat Anti-Human LAG-3 Polyclonal Antibody (R&D Systems), Rabbit Anti-Human LAG-3 Monoclonal Antibody (ab209236) (Abcam), Mouse Anti-Mouse LAG3 Monoclonal Antibody, clone 4-10-C9 (Merck), Rat Anti-Mouse CD223 (LAG-3) Monoclonal Antibody (Invitrogen).

Examples of antibodies for measuring MCP-2 include without limitation: Rat Anti-Mouse CCL8/MCP-2 Monoclonal Antibody (R&D Systems), Mouse Anti-Human CCL8 (MCP-2) Monoclonal Antibody (Invitrogen), Mouse Anti-Human CCL8/MCP-2 Monoclonal Antibody (Novus Biologicals), Rat Anti-Mouse CCL8 (MCP-2) Monoclonal Antibody (BioLegend).

Examples of antibodies for measuring TNFB include without limitation: Recombinant Mouse Anti-Human Lymphotoxin-α/TNF-β Monoclonal Antibody (R&D Systems), Rabbit Anti-Human TNF beta Ployclonal Antibody (Invitrogen), Rabbit Anti-Human TNF beta Polyclonal Antibody (GeneTex).

Examples of antibodies for measuring LILRB4 include without limitation: Rabbit Anti-Human LILRB4 Ployclonal Antibody (Invitrogen), Mouse Anti-Human LILRB4/CD85k/ILT3 Monoclonal Antibody (R&D Systems), Rabbit Anti-Human LILRB4/Gp49 Polyclonal Antibody (Boster Bio), Rabbit Anti-Human ILT3/LILRB4 Polyclonal Antibody (LifeSpan BioSciences).

Examples of antibodies for measuring CKAP4 include without limitation: Sheep Anti-Human CKAP4/p63 Polyclonal Antibody (R&D Systems), Rabbit Anti-Human CKAP4 Ployclonal Antibody (Invitrogen), Rabbit Anti-Human CKAP4 Polyclonal Antibody (Merck).

Examples of antibodies for measuring AREG include without limitation: Rabbit Anti-Human Amphiregulin Polyclonal Antibody (ab180722) (Abcam), Rabbit Anti-Mouse Amphiregulin/AREG Polyclonal Antibody (Boster Bio), Goat Anti-Mouse Amphiregulin Polyclonal Antibody (R&D Systems), Rabbit Anti-Human Amphiregulin Ployclonal Antibody (Invitrogen).

Examples of antibodies for measuring OPG include without limitation: Mouse Anti-Human Osteoprotegerin Monoclonal Antibody (40938) (ThermoFisher), Rabbit Anti-Human Monoclonal Antibody to Osteoprotegerin (ab124820) (Abcam), Rabbit Anti-Human Polyclonal Antibody to Osteoprotegerin (ab73400) (Abcam), Rabbit Anti-Human Polyclonal Antibody to Osteoprotegerin (5312) (BioVision).

Examples of antibodies for measuring FGF23 include without limitation: Goat Anti-Human Polyclonal Antibody to FGF-23 (F2604) (R&D Systems), Goat Anti-Human Polyclonal Antibody to FGF 23 (ab56326) (Abcam), Mouse anti-Human Monoclonal Antibody to FGF23 (LS-C731228) (LSBio), Mouse anti-Human Monoclonal Antibody to FGF 23 (ab190702) (Abcam).

Examples of antibodies for measuring PSIP1 include without limitation: Rabbit Anti-Human PSIP1/LEDGF Monoclonal Antibody (ab177159) (Abcam), Rabbit Anti-Human PSIP1 Monoclonal Antibody (Invitrogen), Rabbit Anti-Human PSIP1 Polyclonal Antibody (Merck).

Examples of antibodies for measuring DCBLD2 include without limitation: Rabbit Anti-Human DCBLD2 Polyclonal Antibody (Invitrogen), Rabbit Anti-Human DCBLD2/ESDN Polyclonal Antibody (Novus Biologicals), Rabbit Anti-Human DCBLD2/ESDN Polyclonal Antibody (ab115451) (Abcam), Sheep Anti-Mouse DCBLD2/ESDN Polyclonal Antibody (R&D Systems).

Examples of antibodies for measuring CLEC7A include without limitation: Rabbit Anti-Human Dectin-1/CLEC7A Polyclonal Antibody (Novus Biologicals), Goat Anti-Mouse Dectin-1/CLEC7A Polyclonal Antibody (R&D Systems), Mouse Anti-Human CD369 (Clec7a, Dectin-1) Monoclonal Antibody (Invitrogen).

Examples of antibodies for measuring EN-RAGE include without limitation: Goat Anti-Human EN-RAGE/S100A12 Polyclonal Antibody (R&D Systems), Rabbit Anti-Human EN-RAGE/S100A12 Polyclonal Antibody (Novus Biologicals), Goat Anti-Human EN-RAGE Polyclonal Antibody (Merck), Rabbit Anti-Human RAGE Polyclonal Antibody (ab3611) (Abcam).

Examples of antibodies for measuring IL8 include without limitation: Mouse Anti-Human IL-8 Monoclonal Antibody (ab18672) (Abcam), Mouse Anti-Human IL-8/CXCL8 Monoclonal Antibody (R&D Systems), Mouse Anti-Human IL-8 (CXCL8) Monoclonal Antibody (Invitrogen).

Examples of antibodies for measuring IL-6 include without limitation: Mouse anti-human IL-6 monoclonal antibody (Clone #1936) (MAB2061) (R&D Systems), Mouse anti-human IL-6 monoclonal antibody (ab9324) (Abcam), Rat anti-human IL-6 monoclonal antibody (MQ2-39C3) (501204) (BioLegend), Rabbit anti-human IL-6 monoclonal antibody (ab233706) (Abcam).

Examples of antibodies for measuring MCP-3 include without limitation: Mouse Anti-Human CCL7 (MCP-3) Monoclonal Antibody (BioLegend), Goat Anti-Mouse CCL7/MCP-3/MARC Polyclonal Antibody (R&D Systems), Rabbit Anti-Human CCL7/MCP-3/MARC Polyclonal Antibody (Novus Biologicals), Mouse Anti-Human MCP-3 Monoclonal Antibody (Invitrogen).

Examples of antibodies for measuring KRT-19 include without limitation: Mouse Anti-Human Monoclonal Antibody to Cytokeratin 19 (ab220193) (Abcam), Rabbit Anti-Human Polyclonal Antibody to KRT19 (LS-C449972) (LSBio), Sheep Anti-Human Polyclonal Antibody to Cytokeratin 19 (AF3506) (R&D Systems), Mouse Anti-Human Monoclonal Antibody to KRT19 (OTI6A8) (ThermoFisher).

Examples of antibodies for measuring LIF (SEQ ID NO: 24) include without limitation: Goat Anti-Human Polyclonal Antibody to LIF (AF-250) (R&D Systems), Mouse Anti-Human Polyclonal Antibody to LIF (ab172023) (Abcam), Mouse Anti-Human Monoclonal Antibody to LIF (MA5-23809) (ThermoFisher), Rabbit Anti-Human Polyclonal Antibody to LIF (ab113262) (Abcam).

Examples of antibodies for measuring CCL-20 include without limitation: Mouse Anti-Human Monoclonal Antibody to CCL20/MIP-3 alpha (MAB360) (R&D Systems), Rabbit Anti-Human Polyclonal Antibody to CCL20/MIP-3 alpha (PA5-114709) (ThermoFisher), Mouse Anti-Human Monoclonal Antibody to CCL20/MIP-3 alpha (LS-C130598) (LSBio), Rabbit Anti-Human Polyclonal Antibody to CCL20/MIP-3 alpha (PA5-95917) (ThermoFisher).

Examples of antibodies for measuring IL-18R1 include without limitation: Rabbit Anti-Human IL-18R1 Polyclonal Antibody (ab117432) (Abcam), Goat Anti-Mouse IL18R1 Polyclonal Antibody (Invitrogen), Mouse Anti-Human IL-18 Ra/1L-1 R5 Monoclonal Antibody (R&D Systems), Rabbit Anti-Human IL18R1 Polyclonal Antibody (Merck).

Examples of antibodies for measuring IL10 include without limitation: Mouse Anti-Human Monoclonal Antibody to IL-10 (MAB217) (R&D Systems), Rat Anti-Human Monoclonal Antibody to IL-10 (14-7108-81) (ThermoFisher), Rabbit Anti-Human Polyclonal Antibody to IL-10 (ab34843) (Abcam), Rabbit Anti-Human Polyclonal Antibody to IL-10 (AHP795) (Bio-Rad).

Examples of antibodies for measuring STC1 include without limitation: Goat Anti-Human Polyclonal Antibody to Stanniocalcin 1/STC-1 (AF2958) (R&D Systems), Rabbit Anti-Human Polyclonal Antibody to STC1 (PA5-35990) (ThermoFisher), Rabbit Anti-Human Polyclonal Antibody to STC1 (ab229477) (Abcam).

Examples of antibodies for measuring HNMT include without limitation: Sheep Anti-Human Polyclonal Antibody to Histamine N-Methyltransferase/HNMT (AF7637) (R&D Systems), Rabbit Anti-Human Polyclonal Antibody to Histamine N-Methyltransferase/HNMT (NBP1-69134) (Novus Biologicals), Mouse Anti-Human Monoclonal Antibody to HNMT (Cat #102-10253) (RayBiotech).

Examples of antibodies for measuring PRDX1 include without limitation: Mouse Anti-Human Monoclonal Antibody to PRDX1 (LF-MA0068) (ThermoFisher), Rabbit Anti-Human Polyclonal Antibody to PRDX1 (PA3-750) (ThermoFisher), Rabbit Anti-Human Polyclonal Antibody to PRDX1 (HPA007730) (Sigma-Aldrich).

Examples of antibodies for measuring HEXIM1 include without limitation: Sheep Anti-Human Polyclonal Antibody to HEXIM1 (AF8106) (R&D Systems), Rabbit Anti-Human Polyclonal Antibody to HEXIM1 (ab25388) (Abcam), Rabbit Anti-Human Polyclonal Antibody to HEXIM1 (#PA5-52629) (ThermoFisher), Goat Anti-Human Polyclonal Antibody to HEXIM1 (VPA00125) (Bio-Rad).

Antibodies suitable for measuring TRAIL include without limitation: Mouse, Monoclonal (55B709-3) IgG (Thermo Fisher Scientific); Mouse, Monoclonal (2E5) IgG1 (Enzo Lifesciences); Mouse, Monoclonal (2E05) IgG1; Mouse, Monoclonal (M912292) IgG1 kappa (My BioSource); Mouse, Monoclonal (IIIF6) IgG2b; Mouse, Monoclonal (2E1-1B9) IgG1 (EpiGentek); Mouse, Monoclonal (RIK-2) IgG1, kappa (BioLegend); Mouse, Monoclonal M181 IgG1 (Immunex Corporation); Mouse, Monoclonal VI10E IgG2b (Novus Biologicals); Mouse, Monoclonal MAB375 IgG1 (R&D Systems); Mouse, Monoclonal MAB687 IgG1 (R&D Systems); Mouse, Monoclonal HS501 IgG1 (Enzo Lifesciences); Mouse, Monoclonal clone 75411.11 Mouse IgG1 (Abcam); Mouse, Monoclonal T8175-50 IgG (X-Zell Biotech Co); Mouse, Monoclonal 2B2.108 IgG1; Mouse, Monoclonal B-T24 IgG1 (Cell Sciences); Mouse, Monoclonal 55B709.3 IgG1 (Thermo Fisher Scientific); Mouse, Monoclonal D3 IgG1 (Thermo Fisher Scientific); Goat, Polyclonal C19 IgG; Rabbit, Polyclonal H257 IgG (Santa Cruz Biotechnology); Mouse, Monoclonal 500-M49 IgG; Mouse, Monoclonal 05-607 IgG; Mouse, Monoclonal B-T24 IgG1 (Thermo Fisher Scientific); Rat, Monoclonal (N2B2), IgG2a, kappa (Thermo Fisher Scientific); Mouse, Monoclonal (1A7-2B7), IgG1 (Genxbio); Mouse, Monoclonal (55B709.3), IgG (Thermo Fisher Scientific); Mouse, Monoclonal B-S23* IgG1 (Cell Sciences), Human TRAIL/TNFSF10 MAb (Clone 75411), Mouse IgG1 (R&D Systems); Human TRAIL/TNFSF10 MAb (Clone 124723), Mouse IgG1 (R&D Systems) and Human TRAIL/TNFSF10 MAb (Clone 75402), Mouse IgG1 (R&D Systems).

Antibodies suitable for measuring CRP include without limitation: Rabbit anti-Human C-Reactive Protein/CRP polyclonal antibody (ab31156) (Abcam), Sheep anti-Human C-Reactive Protein/CRP Polyclonal antibody (AF1707) (R&D Systems), rabbit anti-Human C-Reactive Protein/CRP Polyclonal antibody (C3527) (Sigma-Aldrich), Mouse anti-Human C-Reactive Protein/CRP monoclonal antibody (C1688) (MilliporeSigma).

Antibodies suitable for measuring IP-10 include without limitation: Mouse anti-human CXCL10 (IP-10) Monoclonal Antibody (Cat. No. 524401) (BioLegend), Rabbit anti-human CXCL10 (IP-10) polyclonal Antibody (ab9807) (Abcam), Mouse anti-human CXCL10 (IP-10) Monoclonal Antibody (4D5) (MCA1693) (Bio-Rad), Goat anti-human CXCL10 (IP-10) Monoclonal Antibody (PA5-46999) (Invitrogen), Mouse anti-human CXCL10 (IP-10) Monoclonal Antibody (MA5-23819) (Invitrogen).

Methods of measuring RNA are described herein below.

A “sample” in the context of the present invention is a biological sample isolated from a subject and can include, by way of example and not limitation, whole blood, serum, plasma, saliva, mucus, breath, urine, CSF, sputum, sweat, stool, hair, seminal fluid, biopsy, rhinorrhea, tissue biopsy, cytological sample, platelets, reticulocytes, leukocytes, epithelial cells, or whole blood cells.

In a particular embodiment, the sample is a blood sample—e.g. serum, plasma, or whole blood. The sample may be a venous sample, peripheral blood mononuclear cell sample or a peripheral blood sample. In one embodiment, the sample comprises white blood cells including for example granulocytes, lymphocytes and/or monocytes. In one embodiment, the sample is depleted of red blood cells.

Methods of preparing samples for RNA analysis are described herein below.

The sample is preferably derived from the subject no more than 72 hours, no more than 60 hours, no more than 48 hours, no more than 36 hours, no more than one 24 hours or even no more than 12 hours following symptom onset.

The methods of the present invention are used to classify infections on the basis of severity and etiology (bacterial or viral).

The bacterial or viral infection may be an acute or chronic infection.

A chronic infection is an infection that develops slowly and lasts a long time. Viruses that may cause a chronic infection include Hepatitis C and HIV. One difference between acute and chronic infection is that during acute infection the immune system often produces IgM+ antibodies against the infectious agent, whereas the chronic phase of the infection is usually characteristic of IgM−/IgG+ antibodies. In addition, acute infections cause immune mediated necrotic processes while chronic infections often cause inflammatory mediated fibrotic processes and scaring (e.g. Hepatitis C in the liver). Thus, acute and chronic infections may elicit different underlying immunological mechanisms.

According to a particular embodiment, the infection that is ruled in is an acute infection.

Classification of subjects into subgroups according to this aspect of the present invention is preferably done with an acceptable level of clinical or diagnostic accuracy. An “acceptable degree of diagnostic accuracy”, is herein defined as a test or assay (such as the test used in some aspects of the invention) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, 0.80, desirably at least 0.85, more desirably at least 0.875, preferably at least 0.90, more preferably at least 0.925, and most preferably at least 0.95.

Alternatively, the methods determine severity with at least 75% total accuracy, more preferably 80%, 85%, 90%, 95%, 97%, 98%, 99% or greater total accuracy.

Alternatively, the methods determine severity with an MCC larger than 0.2, 0.3, 0.4, 0.5, 0.6, 0.7, 0.8, 0.9 or 1.0.

As mentioned, in one aspect, the method is for classifying a viral disease. The method may be used to distinguish between a virally infected subject and a non-contagious subject (e.g. healthy subject) as well as classifying the severity of the viral disease.

The term “classifying the severity” refers to assignment of the severity of the disease which may in one embodiment, relate to the probability to experience certain adverse events (e.g. death, hospitalization or admission to ICU) to an individual. Thus, the classification may also be used to prognose the outcome of a patient with an infectious disease. Classifying the severity of the disease may be effected on a binary level (severe/non-severe) or may be effected on non-binary level (e.g. based on numerical values, such as severity categories 1, 2, 3 etc.).

In one embodiment, the severity can be classified according to the WHO ordinal scale of disease stratification.

Exemplary viral diseases which may be diagnosed according to the methods described herein are summarized in Table 15.

TABLE 15
Diseases

gastroenteritis
keratoconjunctivitis
pharyngitis
croup
pharyngoconjunctival fever
pneumonia
cystitis
Hand, foot and mouth disease
pleurodynia
aseptic meningitis
pericarditis
myocarditis
infectious mononucleosis
Burkitt's lymphoma
Hodgkin's lymphoma
nasopharyngeal carcinoma
acute hepatitis
chronic hepatitis
hepatic cirrhosis
hepatocellular carcinoma
herpes labialis, cold sores - can recur by latency
gingivostomatitis in children
tonsillitis & pharyngitis in adults
keratoconjunctivitis
Aseptic meningitis
infectious mononucleosis
Cytomegalic inclusion disease
Kaposi sarcoma
multicentric Castleman disease
primary effusion lymphoma
AIDS
influenza
(Reye syndrome)
measles
postinfectious encephalomyelitis
mumps
hyperplastic epithelial lesions (common, flat,
plantar and anogenital warts, laryngeal
papillomas, epidermodysplasia verruciformis
Malignancies for some species
(cervical carcinoma, squamous cell carcinomas)
croup
pneumonia
bronchiolitis
common cold[
poliomyelitis
rabies (fatal encephalitis)
congenital rubella
German measles
chickenpox
herpes zoster
Congenital varicella syndrome

According to a specific embodiment, the viral disease is COVID-19.

Exemplary virus-causing families are summarized in Table 16, herein below.

TABLE 16

| | Baltimore | | |
|---|---|---|---|
| Family | group | Important species | envelopment |
| Adenoviridae | Group I | Adenovirus | non- |
| | (dsDNA) | | enveloped |
| Herpesviridae | Group I | Herpes simplex, type 1, Herpes simplex, type | enveloped |
| | (dsDNA) | 2, Varicella-zoster virus, Epstein-Barr virus, | |
| | | Human cytomegalovirus, Human herpesvirus, | |
| | | type 8 | |
| Papillomaviridae | Group I | Human papillomavirus | non- |
| | (dsDNA) | | enveloped |
| Polyomaviridae | Group I | BK virus, JC virus | non- |
| | (dsDNA) | | enveloped |
| Poxviridae | Group I | Smallpox | enveloped |
| | (dsDNA) | | |
| Hepadnaviridae | Group VII | Hepatitis B virus | enveloped |
| | (dsDNA-RT) | | |
| Parvoviridae | Group II | Parvovirus B19 | non- |
| | (ssDNA) | | enveloped |
| Astroviridae | Group IV | Human astrovirus | non- |
| | (positive-sense | | enveloped |
| | ssRNA) | | |
| Caliciviridae | Group IV | Norwalk virus | non- |
| | (positive-sense | | enveloped |
| | ssRNA) | | |
| Picornaviridae | Group IV | coxsackievirus, hepatitis A virus, poliovirus, | non- |
| | (positive-sense | rhinovirus | enveloped |
| | ssRNA) | | |
| Coronaviridae | Group IV | Severe acute respiratory syndrome virus | enveloped |
| | (positive-sense | | |
| | ssRNA) | | |
| Flaviviridae | Group IV | Hepatitis C virus, yellow fever virus, dengue | enveloped |
| | (positive-sense | virus, West Nile virus, TBE virus | |
| | ssRNA) | | |
| Togaviridae | Group IV | Rubella virus | enveloped |
| | (positive-sense | | |
| | ssRNA) | | |
| Hepeviridae | Group IV | Hepatitis E virus | non- |
| | (positive-sense | | enveloped |
| | ssRNA) | | |
| Retroviridae | Group VI | Human immunodeficiency virus (HIV) | enveloped |
| | (ssRNA-RT) | | |
| Orthomyxoviridae | Group V | Influenza virus | enveloped |
| | (negative- | | |
| | sense ssRNA) | | |
| Arenaviridae | Group V | Lassa virus | enveloped |
| | (negative- | | |
| | sense ssRNA) | | |
| Bunyaviridae | Group V | Crimean-Congo hemorrhagic fever virus, | enveloped |
| | (negative- | Hantaan virus | |
| | sense ssRNA) | | |
| Filoviridae | Group V | Ebola virus, Marburg virus | enveloped |
| | (negative- | | |
| | sense ssRNA) | | |
| Paramyxoviridae | Group V | Measles virus, Mumps virus, Parainfluenza | enveloped |
| | (negative- | virus, Respiratory syncytial virus, | |
| | sense ssRNA) | | |
| Rhabdoviridae | Group V | Rabies virus | enveloped |
| | (negative- | | |
| | sense ssRNA) | | |
| Unassigned | Group V | Hepatitis D | enveloped |
| | (negative- | | |
| | sense ssRNA) | | |
| Reoviridae | Group III | Rotavirus, Orbivirus, Coltivirus, Banna virus | non- |
| | (dsRNA) | | enveloped |

According to another specific embodiment, the virus is Human metapneumovirus, Bocavirus or Enterovirus.

According to another specific embodiment, the virus is RSV, Flu A, Flu B, HCoV or SARS-Cov-2.

Examples of coronaviruses include: human coronavirus 229E, human coronavirus 0C43, SARS-CoV, HCoV NL63, HKU1, MERS-CoV and SARS-CoV-2.

According to a particular embodiment, the coronavirus is SARS-CoV-2.

For classifying a viral disease, in one aspect at least one protein in Table 4 is measured and at least one protein in Table 6 is measured.

In one embodiment, the classification is carried out by generating a score based on the amount of both a protein in Table 4 and a protein in Table 6.

Proteins listed in Table 4 are proteins whose expression is not affected by the severity of the infection but are influenced by the etiology of the infectious agent (virus/bacteria).

Particular proteins listed in Table 4 whose levels may be measured include HSD11B1, LAMP3, LAG3, CXCL11 and MCP-2.

According to a particular embodiment, the protein from Table 4 is LAMP3, LAG3, MCP-2 and/or CXCL11.

HSD11B1 (Uniprot ID P28845-SEQ ID NO: 1)—an upregulation of the amount of HSD11B1 in the sample above a predetermined level is indicative of a viral infection. The predetermined level may be based on the amount of HSD11B1 present in samples derived from subjects known to have bacterial infections. A downregulation of the amount of HSD11B1 in the sample below a predetermined level is indicative of a bacterial infection. The predetermined level may be based on the amount of HSD11B1 present in samples derived from subjects known to have viral infections.

LAMP3 (Uniprot ID Q9UQV4-SEQ ID NO: 2)—an upregulation of the amount of LAMP3 in the sample above a predetermined level is indicative of a viral infection. The predetermined level may be based on the amount of LAMP3 present in samples derived from subjects known to have bacterial infections.

LAG3 (Uniprot ID P18627-SEQ ID NO: 3)—an upregulation of the amount of LAG3 in the sample above a predetermined level is indicative of a viral infection. The predetermined level may be based on the amount of LAG3 present in samples derived from subjects known to have bacterial infections. A downregulation of the amount of LAG3 in the sample below a predetermined level is indicative of a bacterial infection. The predetermined level may be based on the amount of LAG3 present in samples derived from subjects known to have viral infections.

CXCL11 (Uniprot ID 014625-SEQ ID NO: 4)—an upregulation of the amount of CXCL11 in the sample above a predetermined level is indicative of a viral infection. The predetermined level may be based on the amount of CXCL11 present in samples derived from subjects known to have bacterial infections.

MCP-2 (Uniprot ID P80075-SEQ ID NO: 5)—an upregulation of the amount of MCP-2 in the sample above a predetermined level is indicative of a viral infection. The predetermined level may be based on the amount of MCP-2 present in samples derived from subjects known to have bacterial infections. A downregulation of the amount of MCP-2 in the sample below a predetermined level is indicative of a bacterial infection. The predetermined level may be based on the amount of MCP-2 present in samples derived from subjects known to have viral infections.

Proteins listed in Table 6 are proteins whose expression is not affected by the etiology of the infectious agent but are influenced by the severity of the infection.

Particular proteins from Table 6 whose levels may be measured include PSIP1, LILRB4, DCBLD2, AREG, CLEC7A, IL-18R1, TWEAK, IL10, CCL20, STC1, HNMT, PRDX1, HEXIM1, OPG, FGF-23 and IL8.

According to a specific embodiment, the protein from Table 6 is selected from the group: PSIP1, DCBLD2, AREG, IL10, CCL20, STC1, HNMT, PRDX1, HEXIM1, OPG, FGF-23 and IL8.

PSIP1 (Uniprot ID 075475-SEQ ID NO: 6)—an upregulation of the amount of PSIP1 in the sample above a predetermined level is indicative of a severe infection. The predetermined level may be based on the amount of PSIP1 present in samples derived from subjects known to have non-severe infections. A downregulation of the amount of PSIP1 in the sample below a predetermined level is indicative of a non-severe infection. The predetermined level may be based on the amount of PSIP1 present in samples derived from subjects known to have severe infections.

DCBLD2 (Uniprot ID Q96PD2; SEQ ID NO: 7)—an upregulation of the amount of DCBLD2 in the sample above a predetermined level is indicative of a severe infection. The predetermined level may be based on the amount of DCBLD2 present in samples derived from subjects known to have non-severe infections. A downregulation of the amount of DCBLD2 in the sample below a predetermined level is indicative of a non-severe infection. The predetermined level may be based on the amount of DCBLD2 present in samples derived from subjects known to have severe infections.

CLEC7A (Uniprot ID Q9BXN2; SEQ ID NO: 8)—an upregulation of the amount of CLEC7A in the sample above a predetermined level is indicative of a severe infection. The predetermined level may be based on the amount of CLEC7A present in samples derived from subjects known to have non-severe infections. A downregulation of the amount of CLEC7A in the sample below a predetermined level is indicative of a non-severe infection. The predetermined level may be based on the amount of CLEC7A present in samples derived from subjects known to have severe infections.

LILRB4 (Uniprot ID Q8NHJ6; SEQ ID NO: 9)—an upregulation of the amount of LILRB4 in the sample above a predetermined level is indicative of a severe infection. The predetermined level may be based on the amount of LILRB4 present in samples derived from subjects known to have non-severe infections.

AREG (Uniprot ID P15514; SEQ ID NO: 10)—an upregulation of the amount of AREG in the sample above a predetermined level is indicative of a severe infection. The predetermined level may be based on the amount of AREG present in samples derived from subjects known to have non-severe infections.

IL10 (Uniprot ID P22301; SEQ ID NO: 16)—an upregulation of the amount of IL10 in the sample above a predetermined level is indicative of a severe infection. The predetermined level may be based on the amount of IL10 present in samples derived from subjects known to have non-severe infections.

CCL20 (Uniprot ID P78556; SEQ ID NO: 17) an upregulation of the amount of CCL20 in the sample above a predetermined level is indicative of a severe infection. The predetermined level may be based on the amount of CCL20 present in samples derived from subjects known to have non-severe infections.

STC1 (Uniprot ID P52823; SEQ ID NO: 18) an upregulation of the amount of STC1 in the sample above a predetermined level is indicative of a severe infection. The predetermined level may be based on the amount of STC1 present in samples derived from subjects known to have non-severe infections.

HNMT (Uniprot ID P50135; SEQ ID NO: 19) an upregulation of the amount of HNMT in the sample above a predetermined level is indicative of a severe infection. The predetermined level may be based on the amount of HNMT present in samples derived from subjects known to have non-severe infections.

PRDX1 (Uniprot ID Q06830; SEQ ID NO: 20) an upregulation of the amount of PRDX1 in the sample above a predetermined level is indicative of a severe infection. The predetermined level may be based on the amount of PRDX1 present in samples derived from subjects known to have non-severe infections.

HEXIM1 (Uniprot ID 094992; SEQ ID NO: 21) an upregulation of the amount of HEXIM1 in the sample above a predetermined level is indicative of a severe infection. The predetermined level may be based on the amount of HEXIM1 present in samples derived from subjects known to have non-severe infections.

IL-8 (Uniprot ID P10145; SEQ ID NO: 11)—an upregulation of the amount of IL-8 in the sample above a predetermined level is indicative of a severe infection. The predetermined level may be based on the amount of IL-8 present in samples derived from subjects known to have non-severe infections.

IL-18R1 (Uniprot ID Q13478; SEQ ID NO: 12)—an upregulation of the amount of IL-18R1 in the sample above a predetermined level is indicative of a severe infection. The predetermined level may be based on the amount of IL-18R1 present in samples derived from subjects known to have non-severe infections.

TWEAK (Uniprot ID 043508; SEQ ID NO: 13)—a downregulation of the amount of TWEAK in the sample below a predetermined level is indicative of a severe infection. The predetermined level may be based on the amount of TWEAK present in samples derived from subjects known to have non-severe infections.

OPG (Uniprot ID 000300; SEQ ID NO: 22)—an upregulation of the amount of OPG in the sample above a predetermined level is indicative of a severe infection. The predetermined level may be based on the amount of OPG present in samples derived from subjects known to have non-severe infections (e.g. non-severe bacterial infections).

FGF-23 (Uniprot ID Q9GZV9); SEQ ID NO: 23)—an upregulation of the amount of FGF-23 in the sample above a predetermined level is indicative of a severe infection. The predetermined level may be based on the amount of FGF-23 present in samples derived from subjects known to have non-severe infections (e.g. non-severe bacterial infections).

Particular contemplated combinations of Table 4 markers and Table 6 markers include LAG3 and FGF-23; CXCL11 and FGF-23; MCP-2 and FGF-23; LAMP3 and FGF-23; LAG3 and IL10; CXCL11 and IL10; MCP-2 and IL10; LAMP3 and IL10; LAG3 and CCL20; CXCL11 and CCL20; MCP-2 and CCL20; LAMP3 and CCL20; LAG3 and IL8; CXCL11 and IL8; MCP-2 and IL8; LAMP3 and IL8; LAG3 and STC1; CXCL11 and STC1; MCP-2 and STC1; LAMP3 and STC1; LAG3 and HNMT; CXCL11 and HNMT; MCP-2 and HNMT; LAMP3 and HNMT; LAG3 and AREG; CXCL11 and AREG; MCP-2 and AREG; LAMP3 and AREG; LAG3 and OPG; CXCL11 and OPG; MCP-2 and OPG; LAMP3 and OPG; LAG3 and DCBLD2; CXCL11 and DCBLD2; MCP-2 and DCBLD2; LAMP3 and DCBLD2; LAG3 and PRDX1; CXCL11 and PRDX1; MCP-2 and PRDX1; LAMP3 and PRDX1; LAG3 and PSIP1; CXCL11 and PSIP1; MCP-2 and PSIP1; LAMP3 and PSIP1; LAG3 and HEXIM1; CXCL11 and HEXIM1; MCP-2 and HEXIM1; LAMP3 and HEXIM1.

It will be appreciated that in order to carry out the classification, more than one protein from Table 4 may be measured (e.g. 2, 3, 4 or more) and/or more than one protein from Table 6 may be measured (e.g. 2, 3, or 4).

Particular combinations of Table 4 proteins include LAG3 and CXCL11; LAG3 and MCP-2; LAG3 and LAMP3; CXCL11 and MCP-2; CXCL11 and LAMP3; MCP-2 and LAMP3.

Particular combinations of Table 6 proteins include AREG and OPG; AREG and FGF-23; and OPG and FGF-23. Other combinations include FGF-23 and IL10; FGF-23 and CCL20; FGF-23 and IL8; FGF-23 and STC1; FGF-23 and HNMT; FGF-23 and AREG; FGF-23 and OPG; FGF-23 and DCBLD2; FGF-23 and PRDX1; FGF-23 and PSIP1; FGF-23 and HEXIM1; IL10 and CCL20; IL10 and IL8; IL10 and STC1; IL10 and HNMT; 1L10 and AREG; IL10 and OPG; IL10 and DCBLD2; IL10 and PRDX1; IL10 and PSIP1; IL10 and HEXIM1; CCL20 and IL8; CCL20 and STC1; CCL20 and HNMT; CCL20 and AREG; CCL20 and OPG; CCL20 and DCBLD2; CCL20 and PRDX1; CCL20 and PSIP1; CCL20 and HEXIM1; IL8 and STC1; IL8 and HNMT; IL8 and AREG; IL8 and OPG; IL8 and DCBLD2; IL8 and PRDX1; IL8 and PSIP1; IL8 and HEXIM1; STC1 and HNMT; STC1 and AREG; STC1 and OPG; STC1 and DCBLD2; STC1 and PRDX1; STC1 and PSIP1; STC1 and HEXIM1; HNMT and AREG; HNMT and OPG; HNMT and DCBLD2; HNMT and PRDX1; HNMT and PSIP1; HNMT and HEXIM1; AREG and OPG; AREG and DCBLD2; AREG and PRDX1; AREG and PSIP1; AREG and HEXIM1; OPG and DCBLD2; OPG and PRDX1; OPG and PSIP1; OPG and HEXIM1; DCBLD2 and PRDX1; DCBLD2 and PSIP1; DCBLD2 and HEXIM1; PRDX1 and PSIP1; PRDX1 and HEXIM1; PSIP1 and HEXIM1.

Typically scores based on the amounts of these proteins may be generated that take into account the weights of each of the proteins, as further described herein below.

Bacterial infections which may be ruled in according to embodiments of the invention may be the result of gram-positive, gram-negative bacteria or atypical bacteria.

The term “Gram-positive bacteria” refers to bacteria that are stained dark blue by Gram staining. Gram-positive organisms are able to retain the crystal violet stain because of the high amount of peptidoglycan in the cell wall.

The term “Gram-negative bacteria” refers to bacteria that do not retain the crystal violet dye in the Gram staining protocol.

The term “Atypical bacteria” are bacteria that do not fall into one of the classical “Gram” groups. They are usually, though not always, intracellular bacterial pathogens. They include, without limitations, Mycoplasmas spp., Legionella spp. Rickettsiae spp., and Chlamydiae spp.

For classifying a bacterial disease at least one protein in Table 3 is measured and at least one protein in Table 6 is measured.

In one embodiment, the classification is carried out by generating a score based on the amount of both a protein in Table 3 and a protein in Table 6.

Proteins listed in Table 3 are proteins whose expression is not affected by the severity of the infection but are influenced by the etiology of the infectious agent (bacteria/virus).

Particular proteins listed in Table 3 whose levels may be measured include:

OSM (Uniprot ID P13725; SEQ ID NO: 14)—an upregulation of the amount of OSM in the sample above a predetermined level is indicative of a bacterial infection. In one embodiment, the subject being tested does not show signs of a severe infection. The predetermined level may be based on the amount of OSM present in samples derived from subjects known to have viral infections or known to be non-infected.

Another example is CCL25 (Uniprot ID 015444; SEQ ID NO: 15)—a downregulation of the amount of CCL25 in the sample below a predetermined level is indicative of a bacterial infection. In one embodiment, the subject being tested does not show signs of a severe infection. The predetermined level may be based on the amount of CCL25 present in samples derived from subjects known to have viral infections or known to be non-infected.

Examples of particular combinations of markers (at least one from Table 3 and at least one from Table 6) contemplated by the present invention include: OSM and FGF-23; OSM and IL10; OSM and CCL20; OSM and IL8; OSM and STC1; OSM and HNMT; OSM and AREG; OSM and OPG; OSM and DCBLD2; OSM and PRDX1; OSM and PSIP1; OSM and HEXIM1;

It will be appreciated that in order to carry out the classification, more than one protein from Table 3 may be measured (e.g. 2, 3, 4 or more) and/or more than one protein from Table 6 may be measured (e.g. 2, 3, or 4). Typically scores based on the amounts of these proteins may be generated that take into account the weights of each of the proteins, as further described herein below.

As mentioned, the present inventors also contemplate measuring at least one protein (e.g. 1, 2, 3, 4 or more) which can be used to rule in a bacterial infection (one which appears in Table 3), at least one (1, 2, 3, 4 or more) protein which can be used to rule in a viral infection (one which appears in Table 4) and at least one (1, 2, 3, or 4) protein which can be used to determine the severity of the disease (one that appears in Table 6).

Exemplary combinations include but are not limited to OSM, LAG3 and FGF-23; OSM, CXCL11 and FGF-23; OSM, MCP-2 and FGF-23; OSM, LAMP3 and FGF-23; OSM, LAG3 and IL10; OSM, CXCL11 and IL10; OSM, MCP-2 and IL10; OSM, LAMP3 and IL10; OSM, LAG3 and CCL20; OSM, CXCL11 and CCL20; OSM, MCP-2 and CCL20; OSM, LAMP3 and CCL20; OSM, LAG3 and IL8; OSM, CXCL11 and IL8; OSM, MCP-2 and IL8; OSM, LAMP3 and IL8; OSM, LAG3 and STC1; OSM, CXCL11 and STC1; OSM, MCP-2 and STC1; OSM, LAMP3 and STC1; OSM, LAG3 and HNMT; OSM, CXCL11 and HNMT; OSM, MCP-2 and HNMT; OSM, LAMP3 and HNMT; OSM, LAG3 and AREG; OSM, CXCL11 and AREG; OSM, MCP-2 and AREG; OSM, LAMP3 and AREG; OSM, LAG3 and OPG; OSM, CXCL11 and OPG; OSM, MCP-2 and OPG; OSM, LAMP3 and OPG; OSM, LAG3 and DCBLD2; OSM, CXCL11 and DCBLD2; OSM, MCP-2 and DCBLD2; OSM, LAMP3 and DCBLD2; OSM, LAG3 and PRDX1; OSM, CXCL11 and PRDX1; OSM, MCP-2 and PRDX1; OSM, LAMP3 and PRDX1; OSM, LAG3 and PSIP1; OSM, CXCL11 and PSIP1; OSM, MCP-2 and PSIP1; OSM, LAMP3 and PSIP1; OSM, LAG3 and HEXIM1; OSM, CXCL11 and HEXIM1; OSM, MCP-2 and HEXIM1; OSM, LAMP3 and HEXIM1.

Preferably the combinations which are tested to classify the infectious disease do not exceed 50, 40, 30, 20, 10, 9, 8, 7, 6, 5, 4, 3, or 2 markers. In another embodiment, no more than protein markers are analyzed in a single test/analysis for the classification. In another embodiment, no more than 30 protein markers are analyzed in a single test/analysis for the classification. In another embodiment, no more than 20 protein markers are analyzed in a single test/analysis for the classification. In another embodiment, no more than 10 protein markers are analyzed in a single test/analysis for the classification. In another embodiment, no more than 9 protein markers are analyzed in a single test/analysis for the classification. In another embodiment, no more than 8 protein markers are analyzed in a single test/analysis for the classification. In another embodiment, no more than 7 protein markers are analyzed in a single test/analysis for the classification. In another embodiment, no more than 6 protein markers are analyzed in a single test/analysis, for the classification. In another embodiment, no more than 5 protein markers are analyzed in a single test/analysis for the classification. In another embodiment, no more than 4 protein markers are analyzed in a single test/analysis for the classification. In another embodiment, no more than 3 protein markers are analyzed in a single test/analysis for the classification. In another embodiment, no more than 2 protein markers are analyzed in a single test/analysis for the classification.

According to some embodiments, the classifications are not based on the amount of a protein that is differentially expressed in both (a) bacterial and viral infections; and (b) in severe and non-severe infections. Such proteins include ICA1, IL-24 and CLEC4C.

Additional combinations contemplated for distinguishing between a severe bacterial and a severe viral infection include LIF and TRAIL; CCL20 and TRAIL and FGF-23 and TRAIL.

Still further combinations contemplated for distinguishing between a severe bacterial and a severe viral infection include LIF, TRAIL, CRP and 1P-10; CCL20, TRAIL, CRP and IP10; and FGF-23, TRAIL, CRP and IP-10.

Particular proteins which can be used to distinguish between a severe viral and a non-severe viral are KRT19 (Uniprot No. P08727) and MCP-3 (Uniprot No. P80098).

Thus, if a subject (e.g. one suspected of having a coronaviral disease such as COVID-19) is showing symptoms of the disease, the present inventors contemplate measurement of a combination of these markers to rule in whether the disease is severe/non-severe. Exemplary combinations include KRT19 and MCP-3.

Additional combinations contemplated for distinguishing between a non-severe viral and a severe viral infection include KRT19 and TRAIL; MCP-3 and TRAIL.

Still further combinations contemplated for distinguishing between a severe viral and a non-severe viral infection include KRT19, TRAIL, CRP and IP-10; and MCP-3, TRAIL, CRP and IP-10.

For classifying an infectious disease (according to another aspect) the present inventors contemplate measuring the amount of at least one RNA set forth in Tables 8 or 9 and at least one RNA set forth in Tables 10 or 11 in a sample derived from the subject; and distinguishing between a viral and bacterial disease based on the amount of the at least one RNA set forth in Tables 10 or 11 and determining the severity of the infectious disease based on the amount of the at least one RNA set forth in Tables 8 or 9, thereby classifying the infectious disease.

An increase (beyond a predetermined threshold—e.g. the amount in a patient with a non-severe infection) in the amount of RNA set forth in Table 8 indicates a more severe infection, whereas a decrease (below a predetermined threshold—e.g. the amount in a patient with a non-severe infection) in the amount of RNA set forth in Table 9 indicates a more severe infection.

An increase (beyond a predetermined threshold—e.g. the amount in a healthy subject or a non-infectious subject or a virally infected subject) in the amount of RNA set forth in Table 10 indicates a bacterial infection, whereas a decrease (below a predetermined threshold—e.g. the amount in a healthy subject or a non-infectious subject) in the amount of RNA set forth in Table indicates a viral infection.

An increase (beyond a predetermined threshold—e.g. the amount in a healthy subject or a non-infectious subject or a bacterially infected subject) in the amount of RNA set forth in Table 11 indicates a viral infection, whereas a decrease (below a predetermined threshold—e.g. the amount in a healthy subject or a non-infectious subject) in the amount of RNA set forth in Table 11 indicates a bacterial infection.

In one embodiment, the classification is carried out by generating a score based on the amount of an RNA in Tables 10 or 11 and an RNA in Tables 8 or 9.

On the basis of the classification of the disease, clinical decisions may be made.

According to some embodiments of the invention, the method further comprises informing the subject of results of the classification.

As used herein the phrase “informing the subject” refers to advising the subject that based on the diagnosis the subject should seek a suitable treatment regimen.

Once the diagnosis is determined, the results can be recorded in the subject's medical file, which may assist in selecting a treatment regimen and/or determining prognosis of the subject.

Examples of clinical decisions that may be made in light of a severe classification include oxygen therapy, non-invasive ventilation, mechanical ventilation, invasive monitoring, last-resort drug, sedation, intensive care admission, surgical intervention, hospital admittance, anti-viral drug, antibiotic treatment, anti-viral regimen, anti-fungal drug, immune-globulin treatment, glucocorticoid therapy, extracorporeal membrane oxygenation, kidney replacement therapy.

An example of a clinical decision that may be made in light of a non-severe classification may be isolation.

The antiviral drug may be selected from the group consisting of Remdesivir, Ribavirin, Adefovir, Tenofovir, Acyclovir, Brivudin, Cidofovir, Fomivirsen, Foscarnet, Ganciclovir, Penciclovir, Amantadine, Rimantadine, Zanamivir, Molnupiravir, Paxlovid, Oseltamivir phosphate, Ivermectin, Interferon beta, Interferon alfa, Interferon lambda, Nitazoxanide, Hydroxychloroquine, Peramivir, Baloxavir marboxil, Entecavir, lamivudine and Telbivudine.

Also contemplated are plasma treatments from infected persons who survived and/or anti-HIV drugs such as lopinavir and ritonavir, as well as chloroquine.

Specific examples for drugs that are routinely used for the treatment of COVID-19 include, but are not limited to, Lopinavir/Ritonavir, Nucleoside analogues, Neuraminidase inhibitors, Remdesivir, polypeptide (EK1), abidol, RNA synthesis inhibitors (such as TDF, 3TC), anti-inflammatory drugs (such as hormones and other molecules), Chinese traditional medicine, such ShuFengJieDu Capsules and Lianhuaqingwen Capsule, could be the drug treatment options for COVID19.

According to a specific embodiment, the anti-inflammatory agent is interferon I.

If a bacterial infection is ruled in, the subject may be treated with an antibiotic or other antibacterial agents.

As used herein, the term “antibiotic agent” refers to a group of chemical substances, isolated from natural sources or derived from antibiotic agents isolated from natural sources, having a capacity to inhibit growth of, or to destroy bacteria. Examples of antibiotic agents include, but are not limited to; Amikacin; Amoxicillin; Ampicillin; Azithromycin; Azlocillin; Aztreonam; Aztreonam; Carbenicillin; Cefaclor; Cefepime; Cefetamet; Cefinetazole; Cefixime; Cefonicid; Cefoperazone; Cefotaxime; Cefotetan; Cefoxitin; Cefpodoxime; Cefprozil; Cefsulodin; Ceftazidime; Ceftizoxime; Ceftriaxone; Cefuroxime; Cephalexin; Cephalothin; Cethromycin; Chloramphenicol; Cinoxacin; Ciprofloxacin; Clarithromycin; Clindamycin; Cloxacillin; Co-amoxiclavuanate; Dalbavancin; Daptomycin; Dicloxacillin; Doxycycline; Enoxacin; Erythromycin estolate; Erythromycin ethyl succinate; Erythromycin glucoheptonate; Erythromycin lactobionate; Erythromycin stearate; Erythromycin; Fidaxomicin; Fleroxacin; Gentamicin; Imipenem; Kanamycin; Lomefloxacin; Loracarbef; Methicillin; Metronidazole; Mezlocillin; Minocycline; Mupirocin; Nafcillin; Nalidixic acid; Netilmicin; Nitrofurantoin; Norfloxacin; Ofloxacin; Oxacillin; Penicillin G; Piperacillin; Retapamulin; Rifaxamin, Rifampin; Roxithromycin; Streptomycin; Sulfamethoxazole; Teicoplanin; Tetracycline; Ticarcillin; Tigecycline; Tobramycin; Trimethoprim; Vancomycin; combinations of Piperacillin and Tazobactam; and their various salts, acids, bases, and other derivatives. Anti-bacterial antibiotic agents include, but are not limited to, aminoglycosides, carbacephems, carbapenems, cephalosporins, cephamycins, fluoroquinolones, glycopeptides, linco s amides, macrolides, monobactams, penicillins, quinolones, sulfonamides, and tetracyclines.

Antibacterial agents also include antibacterial peptides. Examples include but are not limited to abaecin; andropin; apidaecins; bombinin; brevinins; buforin II; CAP18; cecropins; ceratotoxin; defensins; dermaseptin; dermcidin; drosomycin; es culentins; indolicidin; LL37; magainin; maximum H5; melittin; moricin; prophenin; protegrin; and or tachyplesins.

Once the classifications are made, additional tests may be made in order to corroborate the result or to further classify the infectious agent.

Examples of such tests include PCR analysis, sequencing analysis, viral culture, antibody or antigen testing.

Performance and Accuracy Measures of the Invention.

The performance and thus absolute and relative clinical usefulness of the invention may be assessed in multiple ways as noted above. Amongst the various assessments of performance, some aspects of the invention are intended to provide accuracy in clinical diagnosis and prognosis. The accuracy of a diagnostic or prognostic test, assay, or method concerns the ability of the test, assay, or method to distinguish between subjects having an infection is based on whether the subjects have, a “significant alteration” (e.g., clinically significant and diagnostically significant) in the levels of a determinant. By “effective amount” it is meant that the measurement of an appropriate number of determinants (which may be one or more) to produce a “significant alteration” (e.g. level of expression or activity of a determinant) that is different than the predetermined cut-off point (or threshold value) for that determinant (s) and therefore indicates that the subject has an infection for which the determinant (s) is an indication. The difference in the level of determinant is preferably statistically significant. As noted below, and without any limitation of the invention, achieving statistical significance, and thus the preferred analytical, diagnostic, and clinical accuracy, may require that combinations of several determinants be used together in panels and combined with mathematical algorithms in order to achieve a statistically significant determinant index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. One way to achieve this is by using the Matthews correlation coefficient (MCC) metric, which depends upon both sensitivity and specificity. Use of statistics such as area under the ROC curve (AUC), encompassing all potential cut point values, is preferred for most categorical risk measures when using some aspects of the invention, while for continuous risk measures, statistics of goodness-of-fit and calibration to observed results or other gold standards, are preferred.

By predetermined level of predictability it is meant that the method provides an acceptable level of clinical or diagnostic accuracy. Using such statistics, an “acceptable degree of diagnostic accuracy”, is herein defined as a test or assay (such as the test used in some aspects of the invention for determining the clinically significant presence of determinants, which thereby indicates the presence an infection type) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, 0.80, desirably at least 0.85, more desirably at least 0.875, preferably at least 0.90, more preferably at least 0.925, and most preferably at least 0.95.

Alternatively, the methods predict the presence or absence of an infection or response to therapy with at least 75% total accuracy, more preferably 80%, 85%, 90%, 95%, 97%, 98%, 99% or greater total accuracy.

Alternatively, the methods predict the presence of a bacterial infection or response to therapy with at least 75% sensitivity, more preferably 80%, 85%, 90%, 95%, 97%, 98%, 99% or greater sensitivity.

Alternatively, the methods predict the presence of a viral infection or response to therapy with at least 75% specificity, more preferably 80%, 85%, 90%, 95%, 97%, 98%, 99% or greater specificity. Alternatively, the methods predict the presence or absence of an infection or response to therapy with an MCC larger than 0.2, 0.3, 0.4, 0.5, 0.6, 0.7, 0.8, 0.9 or 1.0.

In general, alternative methods of determining diagnostic accuracy are commonly used for continuous measures, when a disease category has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, California).

In general, by defining the degree of diagnostic accuracy, i.e., cut points on a ROC curve, defining an acceptable AUC value, and determining the acceptable ranges in relative concentration of what constitutes an effective amount of the determinants of the invention allows for one of skill in the art to use the determinants to identify, diagnose, or prognose subjects with a pre-determined level of predictability and performance.

Furthermore, other unlisted biomarkers will be very highly correlated with the determinants (for the purpose of this application, any two variables will be considered to be “very highly correlated” when they have a Coefficient of Determination (R²) of 0.5 or greater). Some aspects of the present invention encompass such functional and statistical equivalents to the aforementioned determinants. Furthermore, the statistical utility of such additional determinants is substantially dependent on the cross-correlation between multiple biomarkers and any new biomarkers will often be required to operate within a panel in order to elaborate the meaning of the underlying biology.

Construction of Determinant Panels

Groupings of determinants can be included in “panels”, also called “determinant-signatures”, “determinant signatures”, or “multi-determinant signatures.” A “panel” within the context of the present invention means a group of biomarkers (whether they are determinants, clinical parameters, or traditional laboratory risk factors) that includes one or more determinants. A panel can also comprise additional biomarkers, e.g., clinical parameters, traditional laboratory risk factors, known to be present or associated with infection, in combination with a selected group of the determinants listed herein.

As noted above, many of the individual determinants, clinical parameters, and traditional laboratory risk factors listed, when used alone and not as a member of a multi-biomarker panel of determinants, have little or no clinical use in reliably distinguishing individual normal subjects, subjects at risk for having an infection (e.g., bacterial, viral or co-infection), and thus cannot reliably be used alone in classifying any subject between those three states. Even where there are statistically significant differences in their mean measurements in each of these populations, as commonly occurs in studies which are sufficiently powered, such biomarkers may remain limited in their applicability to an individual subject, and contribute little to diagnostic or prognostic predictions for that subject. A common measure of statistical significance is the p-value, which indicates the probability that an observation has arisen by chance alone; preferably, such p-values are 0.05 or less, representing a 5% or less chance that the observation of interest arose by chance. Such p-values depend significantly on the power of the study performed.

Despite this individual determinant performance, and the general performance of formulas combining only the traditional clinical parameters and few traditional laboratory risk factors, the present inventors have noted that certain specific combinations of two or more determinants can also be used as multi-biomarker panels comprising combinations of determinants that are known to be involved in one or more physiological or biological pathways, and that such information can be combined and made clinically useful through the use of various formulae, including statistical classification algorithms and others, combining and in many cases extending the performance characteristics of the combination beyond that of the individual determinants. These specific combinations show an acceptable level of diagnostic accuracy, and, when sufficient information from multiple determinants is combined in a trained formula, they often reliably achieve a high level of diagnostic accuracy transportable from one population to another.

The general concept of how two less specific or lower performing determinants are combined into novel and more useful combinations for the intended indications, is a key aspect of some embodiments of the invention. Multiple biomarkers can yield significant improvement in performance compared to the individual components when proper mathematical and clinical algorithms are used; this is often evident in both sensitivity and specificity, and results in a greater AUC or MCC. Significant improvement in performance could mean an increase of 1%, 2%, 3%, 4%, 5%, 8%, 10% or higher than 10% in different measures of accuracy such as total accuracy, AUC, MCC, sensitivity, specificity, PPV or NPV. Secondly, there is often novel unperceived information in the existing biomarkers, as such was necessary in order to achieve through the new formula an improved level of sensitivity or specificity. This hidden information may hold true even for biomarkers which are generally regarded to have suboptimal clinical performance on their own. In fact, the suboptimal performance in terms of high false positive rates on a single biomarker measured alone may very well be an indicator that some important additional information is contained within the biomarker results—information which would not be elucidated absent the combination with a second biomarker and a mathematical formula.

On the other hand, it is often useful to restrict the number of measured diagnostic determinants (e.g., protein markers), as this allows significant cost reduction and reduces required sample volume and assay complexity. Accordingly, even when two signatures have similar diagnostic performance (e.g., similar AUC or sensitivity), one which incorporates less proteins could have significant utility and ability to reduce to practice. For example, a signature that includes 5 proteins compared to 10 proteins and performs similarly has many advantages in real world clinical setting and thus is desirable. Therefore, there is value and invention in being able to reduce the number of proteins incorporated in a signature while retaining similar levels of accuracy. In this context similar levels of accuracy could mean plus or minus 1%, 2%, 3%, 4%, 5%, 8%, or 10% in different measures of accuracy such as total accuracy, AUC, MCC, sensitivity, specificity, PPV or NPV; a significant reduction in the number of proteins of a signature includes reducing the number of proteins by 2, 3, 4, 5, 6, 7, 8, 9, 10 or more than 10 proteins.

Several statistical and modeling algorithms known in the art can be used to both assist in determinant selection choices and optimize the algorithms combining these choices. Statistical tools such as factor and cross-biomarker correlation/covariance analyses allow more rationale approaches to panel construction. Mathematical clustering and classification tree showing the Euclidean standardized distance between the determinants can be advantageously used. Pathway informed seeding of such statistical classification techniques also may be employed, as may rational approaches based on the selection of individual determinants based on their participation across in particular pathways or physiological functions.

Ultimately, formula such as statistical classification algorithms can be directly used to both select determinants and to generate and train the optimal formula necessary to combine the results from multiple determinants into a single index. Often, techniques such as forward (from zero potential explanatory parameters) and backwards selection (from all available potential explanatory parameters) are used, and information criteria, such as AIC or BIC, are used to quantify the tradeoff between the performance and diagnostic accuracy of the panel and the number of determinants used. The position of the individual determinant on a forward or backwards selected panel can be closely related to its provision of incremental information content for the algorithm, so the order of contribution is highly dependent on the other constituent determinants in the panel.

Construction of Clinical Algorithms

Any formula may be used to combine determinant results into indices useful in the practice of the invention. As indicated above, and without limitation, such indices may indicate, among the various other indications, the probability, likelihood, absolute or relative risk, time to or rate of conversion from one to another disease states, or make predictions of future biomarker measurements of infection. This may be for a specific time period or horizon, or for remaining lifetime risk, or simply be provided as an index relative to another reference subject population.

Although various preferred formula are described here, several other model and formula types beyond those mentioned herein and in the definitions above are well known to one skilled in the art. The actual model type or formula used may itself be selected from the field of potential models based on the performance and diagnostic accuracy characteristics of its results in a training population. The specifics of the formula itself may commonly be derived from determinant results in the relevant training population. Amongst other uses, such formula may be intended to map the feature space derived from one or more determinant inputs to a set of subject classes (e.g. useful in predicting class membership of subjects as normal, having an infection), to derive an estimation of a probability function of risk using a Bayesian approach, or to estimate the class-conditional probabilities, then use Bayes' rule to produce the class probability function as in the previous case.

Preferred formulas include the broad class of statistical classification algorithms, and in particular the use of discriminant analysis. The goal of discriminant analysis is to predict class membership from a previously identified set of features. In the case of linear discriminant analysis (LDA), the linear combination of features is identified that maximizes the separation among groups by some criteria. Features can be identified for LDA using an eigengene based approach with different thresholds (ELDA) or a stepping algorithm based on a multivariate analysis of variance (MANOVA). Forward, backward, and stepwise algorithms can be performed that minimize the probability of no separation based on the Hotelling-Lawley statistic.

Eigengene-based Linear Discriminant Analysis (ELDA) is a feature selection technique developed by Shen et al. (2006). The formula selects features (e.g. biomarkers) in a multivariate framework using a modified eigen analysis to identify features associated with the most important eigenvectors. “Important” is defined as those eigenvectors that explain the most variance in the differences among samples that are trying to be classified relative to some threshold.

A support vector machine (SVM) is a classification formula that attempts to find a hyperplane that separates two classes. This hyperplane contains support vectors, data points that are exactly the margin distance away from the hyperplane. In the likely event that no separating hyperplane exists in the current dimensions of the data, the dimensionality is expanded greatly by projecting the data into larger dimensions by taking non-linear functions of the original variables (Venables and Ripley, 2002). Although not required, filtering of features for SVM often improves prediction. Features (e.g., biomarkers) can be identified for a support vector machine using a non-parametric Kruskal-Wallis (KW) test to select the best univariate features. A random forest (RF, Breiman, 2001) or recursive partitioning (RPART, Breiman et al., 1984) can also be used separately or in combination to identify biomarker combinations that are most important. Both KW and RF require that a number of features be selected from the total. RPART creates a single classification tree using a subset of available biomarkers.

Other formula may be used in order to pre-process the results of individual determinant measurements into more valuable forms of information, prior to their presentation to the predictive formula. Most notably, normalization of biomarker results, using either common mathematical transformations such as logarithmic or logistic functions, as normal or other distribution positions, in reference to a population's mean values, etc. are all well known to those skilled in the art. Of particular interest are a set of normalizations based on clinical-determinants such as time from symptoms, gender, race, or sex, where specific formula are used solely on subjects within a class or continuously combining a clinical-determinants as an input. In other cases, analyte-based biomarkers can be combined into calculated variables which are subsequently presented to a formula.

In addition to the individual parameter values of one subject potentially being normalized, an overall predictive formula for all subjects, or any known class of subjects, may itself be recalibrated or otherwise adjusted based on adjustment for a population's expected prevalence and mean biomarker parameter values, according to the technique outlined in D'Agostino et al., (2001) JAMA 286:180-187, or other similar normalization and recalibration techniques. Such epidemiological adjustment statistics may be captured, confirmed, improved and updated continuously through a registry of past data presented to the model, which may be machine readable or otherwise, or occasionally through the retrospective query of stored samples or reference to historical studies of such parameters and statistics. Additional examples that may be the subject of formula recalibration or other adjustments include statistics used in studies by Pepe, M. S. et al., 2004 on the limitations of odds ratios; Cook, N. R., 2007 relating to ROC curves. Finally, the numeric result of a classifier formula itself may be transformed post-processing by its reference to an actual clinical population and study results and observed endpoints, in order to calibrate to absolute risk and provide confidence intervals for varying numeric results of the classifier or risk formula.

Some determinants may exhibit trends that depends on the patient age (e.g. the population baseline may rise or fall as a function of age). One can use a ‘Age dependent normalization or stratification’ scheme to adjust for age related differences. Performing age dependent normalization, stratification or distinct mathematical formulas can be used to improve the accuracy of determinants for differentiating between different types of infections. For example, one skilled in the art can generate a function that fits the population mean levels of each determinant as function of age and use it to normalize the determinant of individual subjects levels across different ages. Another example is to stratify subjects according to their age and determine age specific thresholds or index values for each age group independently.

In the context of the present invention the following statistical terms may be used:

“TP” is true positive, means positive test result that accurately reflects the tested-for activity. For example in the context of the present invention a TP, is for example but not limited to, truly classifying a bacterial infection as such.

“TN” is true negative, means negative test result that accurately reflects the tested-for activity. For example in the context of the present invention a TN, is for example but not limited to, truly classifying a viral infection as such.

“FN” is false negative, means a result that appears negative but fails to reveal a situation. For example in the context of the present invention a FN, is for example but not limited to, falsely classifying a bacterial infection as a viral infection.

“FP” is false positive, means test result that is erroneously classified in a positive category. For example in the context of the present invention a FP, is for example but not limited to, falsely classifying a viral infection as a bacterial infection.

“Sensitivity” is calculated by TP/(TP+FN) or the true positive fraction of disease subjects.

“Specificity” is calculated by TN/(TN+FP) or the true negative fraction of non-disease or normal subjects.

“Total accuracy” is calculated by (TN+TP)/(TN+FP+TP+FN).

“Positive predictive value” or “PPV” is calculated by TP/(TP+FP) or the true positive fraction of all positive test results. It is inherently impacted by the prevalence of the disease and pre-test probability of the population intended to be tested.

“Negative predictive value” or “NPV” is calculated by TN/(TN+FN) or the true negative fraction of all negative test results. It also is inherently impacted by the prevalence of the disease and pre-test probability of the population intended to be tested. See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test.

“MCC” (Mathews Correlation coefficient) is calculated as follows: MCC=(TP*TN−FP*FN)/{(TP+FN)*(TP+FP)*(TN+FP)*(TN+FN)}\hat{ }0.5 where TP, FP, TN, FN are true-positives, false-positives, true-negatives, and false-negatives, respectively. Note that MCC values range between −1 to +1, indicating completely wrong and perfect classification, respectively. An MCC of 0 indicates random classification. MCC has been shown to be a useful for combining sensitivity and specificity into a single metric (Baldi, Brunak et al. 2000). It is also useful for measuring and optimizing classification accuracy in cases of unbalanced class sizes (Baldi, Brunak et al. 2000).

Often, for binary disease state classification approaches using a continuous diagnostic test measurement, the sensitivity and specificity is summarized by a Receiver Operating Characteristics (ROC) curve according to Pepe et al., “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4^{th} edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronory Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), Mathews correlation coefficient (MCC), or as a likelihood, odds ratio, Receiver Operating Characteristic (ROC) curve, Area Under the Curve (AUC) among other measures.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value”. Non-limiting examples of “formulas” include sums, ratios, and regression operators, such as coefficients or exponents, biomarker value transformations and normalizations (including, without limitation, those normalization schemes based on clinical-determinants, such as gender, age, or ethnicity), rules and guidelines, statistical classification models, and neural networks trained on historical populations. Of particular use in combining determinants are linear and non-linear equations and statistical classification analyses to determine the relationship between levels of determinants detected in a subject sample and the subject's probability of having an infection or a certain type of infection. In panel and combination construction, of particular interest are structural and syntactic statistical classification algorithms, and methods of index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, and Hidden Markov Models, among others. Other techniques may be used in survival and time to event hazard analysis, including Cox, Weibull, Kaplan-Meier and Greenwood models well known to those of skill in the art. Many of these techniques are useful either combined with a determinant selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art. A “health economic utility function” is a formula that is derived from a combination of the expected probability of a range of clinical outcomes in an idealized applicable patient population, both before and after the introduction of a diagnostic or therapeutic intervention into the standard of care. It encompasses estimates of the accuracy, effectiveness and performance characteristics of such intervention, and a cost and/or value measurement (a utility) associated with each outcome, which may be derived from actual health system costs of care (services, supplies, devices and drugs, etc.) and/or as an estimated acceptable value per quality adjusted life year (QALY) resulting in each outcome. The sum, across all predicted outcomes, of the product of the predicted population size for an outcome multiplied by the respective outcome's expected utility is the total health economic utility of a given standard of care. The difference between (i) the total health economic utility calculated for the standard of care with the intervention versus (ii) the total health economic utility for the standard of care without the intervention results in an overall measure of the health economic cost or value of the intervention. This may itself be divided amongst the entire patient group being analyzed (or solely amongst the intervention group) to arrive at a cost per unit intervention, and to guide such decisions as market positioning, pricing, and assumptions of health system acceptance. Such health economic utility functions are commonly used to compare the cost-effectiveness of the intervention, but may also be transformed to estimate the acceptable value per QALY the health care system is willing to pay, or the acceptable cost-effective clinical performance characteristics required of a new intervention.

For diagnostic (or prognostic) interventions of the invention, as each outcome (which in a disease classifying diagnostic test may be a TP, FP, TN, or FN) bears a different cost, a health economic utility function may preferentially favor sensitivity over specificity, or PPV over NPV based on the clinical situation and individual outcome costs and value, and thus provides another measure of health economic performance and value which may be different from more direct clinical or analytical performance measures. These different measurements and relative trade-offs generally will converge only in the case of a perfect test, with zero error rate (a.k.a., zero predicted subject outcome misclassifications or FP and FN), which all performance measures will favor over imperfection, but to differing degrees.

“Analytical accuracy” refers to the reproducibility and predictability of the measurement process itself, and may be summarized in such measurements as coefficients of variation (CV), Pearson correlation, and tests of concordance and calibration of the same samples or controls with different times, users, equipment and/or reagents. These and other considerations in evaluating new biomarkers are also summarized in Vasan, 2006.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC and MCC, time to result, shelf life, etc. as relevant.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

Kits

Some aspects of the invention also include a determinant-detection reagent such as antibodies packaged together in the form of a kit. The kit may contain in separate containers antibodies (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. The detectable label may be attached to a secondary antibody which binds to the Fc portion of the antibody which recognizes the determinant. Instructions (e.g., written, tape, VCR, CD-ROM, etc.) for carrying out the assay may be included in the kit.

The kits of this aspect of the present invention may comprise additional components that aid in the detection of the determinants such as enzymes, salts, buffers etc. necessary to carry out the detection reactions.

For example, determinant detection reagents (e.g. antibodies) can be immobilized on a solid support such as a porous strip or an array to form at least one determinant detection site. The measurement or detection region of the porous strip may include a plurality of sites. A test strip may also contain sites for negative and/or positive controls. Alternatively, control sites can be located on a separate strip from the test strip. Optionally, the different detection sites may contain different amounts of immobilized detection reagents, e.g., a higher amount in the first detection site and lesser amounts in subsequent sites. Upon the addition of test sample, the number of sites displaying a detectable signal provides a quantitative indication of the amount of determinants present in the sample. The detection sites may be configured in any suitably detectable shape and are typically in the shape of a bar or dot spanning the width of a test strip.

Polyclonal antibodies for measuring determinants include without limitation antibodies that were produced from sera by active immunization of one or more of the following: Rabbit, Goat, Sheep, Chicken, Duck, Guinea Pig, Mouse, Donkey, Camel, Rat and Horse.

Examples of detection agents, include without limitation: scFv, dsFv, Fab, sVH, F(ab′)2, Cyclic peptides, Haptamers, A single-domain antibody, Fab fragments, Single-chain variable fragments, Affibody molecules, Affilins, Nanofitins, Anticalins, Avimers, DARPins, Kunitz domains, Fynomers and Monobody.

In particular embodiments, the kit does not comprise a number of antibodies that specifically recognize more than 50, 20 15, 10, 9, 8, 7, 6, 5 or 4 polypeptides.

In other embodiments, the array of the present invention does not comprise a number of antibodies that specifically recognize more than 50, 20 15, 10, 9, 8, 7, 6, 5 or 4 polypeptides.

According to a particular embodiment, the kit contains at least one antibody which specifically binds to a determinant listed in Table 4 and at least one antibody which specifically binds to a determinant listed in Table 6.

According to a particular embodiment, the kit contains at least one antibody which specifically binds to a determinant listed in Table 3 and at least one antibody which specifically binds to a determinant listed in Table 6.

According to a particular embodiment, the kit contains at least one antibody which specifically binds to a determinant listed in Table 3, at least one antibody which specifically binds to a determinant listed in Table 4 and at least one antibody which specifically binds to a determinant listed on Table 6.

Particular combinations of antibodies are provided herein above.

In one embodiment, the kit is devoid of antibodies that specifically detect a protein that is differentially expressed in both (a) bacterial and viral infections; and (b) in severe and non-severe infections. Such proteins are listed herein above.

A machine-readable storage medium can comprise a data storage material encoded with machine-readable data or data arrays which, when using a machine programmed with instructions for using the data, is capable of use for a variety of purposes. Measurements of effective amounts of the biomarkers of the invention and/or the resulting evaluation of risk from those biomarkers can be implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein. The health-related data management system used in some aspects of the invention may also be considered to be implemented as a computer-readable storage medium, configured with a computer program, where the storage medium so configured causes a computer to operate in a specific and predefined manner to perform various functions described herein.

The polypeptide determinants of the present invention, in some embodiments thereof, can be used to generate a “reference determinant profile” of those subjects who do not have an infection. The determinants disclosed herein can also be used to generate a “subject determinant profile” taken from subjects who have an infection. The subject determinant profiles can be compared to a reference determinant profile to diagnose or identify subjects with an infection. The subject determinant profile of different infection types can be compared to diagnose or identify the type of infection. The reference and subject determinant profiles of the present invention, in some embodiments thereof, can be contained in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other disease-risk algorithms and computed indices such as those described herein.

RNA Analysis:

Isolation, extraction or derivation of RNA may be carried out by any suitable method. Isolating RNA from a biological sample generally includes treating a biological sample in such a manner that the RNA present in the sample is extracted and made available for analysis. Any isolation method that results in extracted RNA may be used in the practice of the present invention. It will be understood that the particular method used to extract RNA will depend on the nature of the source.

Methods of RNA extraction are well-known in the art and further described herein under.

Phenol based extraction methods: These single-step RNA isolation methods based on Guanidine isothiocyanate (GITC)/phenol/chloroform extraction require much less time than traditional methods (e.g. CsCl₂ ultracentrifugation). Many commercial reagents (e.g. Trizol, RNAzol, RNAWIZ) are based on this principle. The entire procedure can be completed within an hour to produce high yields of total RNA.

Silica gel—based purification methods: RNeasy is a purification kit marketed by Qiagen. It uses a silica gel-based membrane in a spin-column to selectively bind RNA larger than 200 bases. The method is quick and does not involve the use of phenol.

Oligo-dT based affinity purification of mRNA: Due to the low abundance of mRNA in the total pool of cellular RNA, reducing the amount of rRNA and tRNA in a total RNA preparation greatly increases the relative amount of mRNA. The use of oligo-dT affinity chromatography to selectively enrich poly (A)+RNA has been practiced for over 20 years. The result of the preparation is an enriched mRNA population that has minimal rRNA or other small RNA contamination. mRNA enrichment is essential for construction of cDNA libraries and other applications where intact mRNA is highly desirable. The original method utilized oligo-dT conjugated resin column chromatography and can be time consuming. Recently more convenient formats such as spin-column and magnetic bead based reagent kits have become available.

The sample may also be processed prior to carrying out the diagnostic methods of the present invention. Processing of the sample may involve one or more of: filtration, distillation, centrifugation, extraction, concentration, dilution, purification, inactivation of interfering components, addition of reagents, and the like.

After obtaining the RNA sample, cDNA may be generated therefrom. For synthesis of cDNA, template mRNA may be obtained directly from lysed cells or may be purified from a total RNA or mRNA sample. The total RNA sample may be subjected to a force to encourage shearing of the RNA molecules such that the average size of each of the RNA molecules is between 100-300 nucleotides, e.g. about 200 nucleotides. To separate the heterogeneous population of mRNA from the majority of the RNA found in the cell, various technologies may be used which are based on the use of oligo(dT) oligonucleotides attached to a solid support. Examples of such oligo(dT) oligonucleotides include: oligo(dT) cellulose/spin columns, oligo(dT)/magnetic beads, and oligo(dT) oligonucleotide coated plates.

Generation of single stranded DNA from RNA requires synthesis of an intermediate RNA-DNA hybrid. For this, a primer is required that hybridizes to the 3′ end of the RNA. Annealing temperature and timing are determined both by the efficiency with which the primer is expected to anneal to a template and the degree of mismatch that is to be tolerated.

The annealing temperature is usually chosen to provide optimal efficiency and specificity, and generally ranges from about 50° C. to about 80° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

According to a specific embodiment, the primer comprises a polydT oligonucleotide sequence.

Preferably the polydT sequence comprises at least 5 nucleotides. According to another is between about 5 to 50 nucleotides, more preferably between about 5-25 nucleotides, and even more preferably between about 12 to 14 nucleotides.

Following annealing of the primer (e.g. polydT primer) to the RNA sample, an RNA-DNA hybrid is synthesized by reverse transcription using an RNA-dependent DNA polymerase. Suitable RNA-dependent DNA polymerases for use in the methods and compositions of the invention include reverse transcriptases (RTs). Examples of RTs include, but are not limited to, Moloney murine leukemia virus (M-MLV) reverse transcriptase, human immunodeficiency virus (HIV) reverse transcriptase, rous sarcoma virus (RSV) reverse transcriptase, avian myeloblastosis virus (AMV) reverse transcriptase, rous associated virus (RAV) reverse transcriptase, and myeloblastosis associated virus (MAV) reverse transcriptase or other avian sarcoma-leukosis virus (ASLV) reverse transcriptases, and modified RTs derived therefrom. See e.g. U.S. Pat. No. 7,056,716. Many reverse transcriptases, such as those from avian myeloblastosis virus (AMV-RT), and Moloney murine leukemia virus (MMLV-RT) comprise more than one activity (for example, polymerase activity and ribonuclease activity) and can function in the formation of the double stranded cDNA molecules.

Additional components required in a reverse transcription reaction include dNTPS (dATP, dCTP, dGTP and dTTP) and optionally a reducing agent such as Dithiothreitol (DTT) and MnCl₂.

Methods of analyzing the amount of RNA are known in the art and are summarized infra:

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls. Isothermal amplification is also contemplated.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In situ RT-PCR stain: This method is described in Nuovo G J, et al. [Intracellular localization of polymerase chain reaction (PCR)—amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, CA).

DNA Microarrays/DNA Chips:

The expression of thousands of genes may be analyzed simultaneously using DNA microarrays, allowing analysis of the complete transcriptional program of an organism during specific developmental processes or physiological responses. DNA microarrays consist of thousands of individual gene sequences attached to closely packed areas on the surface of a support such as a glass microscope slide. Various methods have been developed for preparing DNA microarrays. In one method, an approximately 1 kilobase segment of the coding region of each gene for analysis is individually PCR amplified. A robotic apparatus is employed to apply each amplified DNA sample to closely spaced zones on the surface of a glass microscope slide, which is subsequently processed by thermal and chemical treatment to bind the DNA sequences to the surface of the support and denature them. Typically, such arrays are about 2×2 cm and contain about individual nucleic acids 6000 spots. In a variant of the technique, multiple DNA oligonucleotides, usually 20 nucleotides in length, are synthesized from an initial nucleotide that is covalently bound to the surface of a support, such that tens of thousands of identical oligonucleotides are synthesized in a small square zone on the surface of the support. Multiple oligonucleotide sequences from a single gene are synthesized in neighboring regions of the slide for analysis of expression of that gene. Hence, thousands of genes can be represented on one glass slide. Such arrays of synthetic oligonucleotides may be referred to in the art as “DNA chips”, as opposed to “DNA microarrays”, as described above [Lodish et al. (eds.). Chapter 7.8: DNA Microarrays: Analyzing Genome-Wide Expression. In: Molecular Cell Biology, 4th ed., W. H. Freeman, New York. (2000)].

Oligonucleotide microarray—In this method oligonucleotide probes capable of specifically hybridizing with the polynucleotides of some embodiments of the invention are attached to a solid surface (e.g., a glass wafer). Each oligonucleotide probe is of approximately 20-25 nucleic acids in length. To detect the expression pattern of the polynucleotides of some embodiments of the invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, MD, USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara CA). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, CA) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

RNA sequencing: Methods for RNA sequence determination are generally known to the person skilled in the art. Preferred sequencing methods are next generation sequencing methods or parallel high throughput sequencing methods. An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods. The present invention also envisages further developments of these techniques, e.g. further improvements of the accuracy of the sequence determination, or the time needed for the determination of the genomic sequence of an organism etc.

According to one embodiment, the sequencing method comprises deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

It will be appreciated that in order to analyze the amount of an RNA marker, oligonucleotides may be used that are capable of hybridizing thereto or to cDNA generated therefrom. According to one embodiment a single oligonucleotide is used to determine the presence of a particular RNA marker, at least two oligonucleotides are used to determine the presence of a particular RNA marker, at least three oligonucleotides are used to determine the presence of a particular RNA marker, at least four oligonucleotides are used to determine the presence of a particular RNA marker, at least five or more oligonucleotides are used to determine the presence of a particular RNA marker.

When more than one oligonucleotide is used, the sequence of the oligonucleotides may be selected such that they hybridize to the same exon of the RNA marker or different exons of the RNA marker. In one embodiment, at least one of the oligonucleotides hybridizes to the 3′ exon of the RNA markder. In another embodiment, at least one of the oligonucleotides hybridizes to the 5′ exon of the RNA marker.

In one embodiment, the method of this aspect of the present invention is carried out using an isolated oligonucleotide which hybridizes to the RNA or cDNA of any of the RNA markers disclosed herein by complementary base-pairing in a sequence specific manner, and discriminates the determinant sequence from other nucleic acid sequence in the sample. Oligonucleotides (e.g. DNA or RNA oligonucleotides) typically comprises a region of complementary nucleotide sequence that hybridizes under stringent conditions to at least about 8, 10, 13, 16, 18, 20, 22, 25, 30, 40, 50, 55, 60, 65, 70, 80, 90, 100, 120 (or any other number in-between) or more consecutive nucleotides in a target nucleic acid molecule. Depending on the particular assay, the consecutive nucleotides include the determinant nucleic acid sequence.

The term “isolated”, as used herein in reference to an oligonucleotide, means an oligonucleotide, which by virtue of its origin or manipulation, is separated from at least some of the components with which it is naturally associated or with which it is associated when initially obtained. By “isolated”, it is alternatively or additionally meant that the oligonucleotide of interest is produced or synthesized by the hand of man.

In order to identify an oligonucleotide specific for any of the RNA markers disclosed herein, the gene/transcript of interest is typically examined using a computer algorithm which starts at the 5′ or at the 3′ end of the nucleotide sequence. Typical algorithms will then identify oligonucleotides of defined length that are unique to the gene, have a GC content within a range suitable for hybridization, lack predicted secondary structure that may interfere with hybridization, and/or possess other desired characteristics or that lack other undesired characteristics.

Following identification of the oligonucleotide it may be tested for specificity towards the determinant under wet or dry conditions. Thus, for example, in the case where the oligonucleotide is a primer, the primer may be tested for its ability to amplify a sequence of the determinant using PCR to generate a detectable product and for its non ability to amplify other determinants in the sample. The products of the PCR reaction may be analyzed on a gel and verified according to presence and/or size.

Additionally, or alternatively, the sequence of the oligonucleotide may be analyzed by computer analysis to see if it is homologous (or is capable of hybridizing to) other known sequences. A BLAST 2.2.10 (Basic Local Alignment Search Tool) analysis may be performed on the chosen oligonucleotide (worldwidewebdotncbidotnlmdotnihdotgov/blast/). The BLAST program finds regions of local similarity between sequences. It compares nucleotide or protein sequences to sequence databases and calculates the statistical significance of matches thereby providing valuable information about the possible identity and integrity of the ‘query’ sequences.

According to one embodiment, the oligonucleotide is a probe. As used herein, the term “probe” refers to an oligonucleotide which hybridizes to the determinant specific nucleic acid sequence to provide a detectable signal under experimental conditions and which does not hybridize to additional determinant sequences to provide a detectable signal under identical experimental conditions.

The probes of this embodiment of this aspect of the present invention may be, for example, affixed to a solid support (e.g., arrays or beads).

Solid supports are solid-state substrates or supports onto which the nucleic acid molecules of the present invention may be associated. The nucleic acids may be associated directly or indirectly. Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

In one embodiment, the solid support is an array which comprises a plurality of nucleic acids which hybridize to RNA markers of the present invention immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

According to particular embodiments, the array does not comprise nucleic acids that specifically bind to more than 50 RNA markers, more than 40 RNA markers, 30 RNA markers, 20 RNA markers, 15 RNA markers, 10 RNA markers, 5 RNA markers or even 3 RNA markers.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

According to another embodiment, the oligonucleotide is a primer of a primer pair. As used herein, the term “primer” refers to an oligonucleotide which acts as a point of initiation of a template-directed synthesis using methods such as PCR (polymerase chain reaction) or LCR (ligase chain reaction) under appropriate conditions (e.g., in the presence of four different nucleotide triphosphates and a polymerization agent, such as DNA polymerase, RNA polymerase or reverse-transcriptase, DNA ligase, etc, in an appropriate buffer solution containing any necessary co-factors and at suitable temperature(s)). Such a template directed synthesis is also called “primer extension”. For example, a primer pair may be designed to amplify a region of DNA using PCR. Such a pair will include a “forward primer” and a “reverse primer” that hybridize to complementary strands of a DNA molecule and that delimit a region to be synthesized/amplified. A primer of this aspect of the present invention is capable of amplifying, together with its pair (e.g. by PCR) a determinant specific nucleic acid sequence to provide a detectable signal under experimental conditions and which does not amplify other determinant nucleic acid sequence to provide a detectable signal under identical experimental conditions.

According to additional embodiments, the oligonucleotide is about 8, 9, 10, 11, 12, 13, 14, 15, 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific preferred embodiment of the invention, a primer or a probe is within the length of about 18 and about 28 nucleotides. It will be appreciated that when attached to a solid support, the probe may be of about 30-70, 75, 80, 90, 100, or more nucleotides in length.

The oligonucleotide of this aspect of the present invention need not reflect the exact sequence of the RNA marker nucleic acid sequence (i.e. need not be fully complementary), but must be sufficiently complementary to hybridize with the determinant nucleic acid sequence under the particular experimental conditions. Accordingly, the sequence of the oligonucleotide typically has at least 70% homology, preferably at least 80%, 90%, 95%, 97%, 99% or 100% homology, for example over a region of at least 13 or more contiguous nucleotides with the target determinant nucleic acid sequence. The conditions are selected such that hybridization of the oligonucleotide to the determinant nucleic acid sequence is favored and hybridization to other determinant nucleic acid sequences is minimized.

By way of example, hybridization of short nucleic acids (below 200 bp in length, e.g. 13-50 bp in length) can be effected by the following hybridization protocols depending on the desired stringency; (i) hybridization solution of 6×SSC and 1% SDS or 3 M TMACl, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tm, final wash solution of 3 M TMACl, 0.01 M sodium phosphate (pH 6.8), 1 mM EDTA (pH 7.6), 0.5% SDS at 1-1.5° C. below the Tm (stringent hybridization conditions) (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tm, final wash solution of 3 M TMACl, 0.01 M sodium phosphate (pH 6.8), 1 mM EDTA (pH 7.6), 0.5% SDS at 1-1.5° C. below the Tm, final wash solution of 6×SSC, and final wash at 22° C. (stringent to moderate hybridization conditions); and (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature at 2.5-3° C. below the Tm and final wash solution of 6×SSC at 22° C. (moderate hybridization solution).

Oligonucleotides of the invention may be prepared by any of a variety of methods (see, for example, J. Sambrook et al., “Molecular Cloning: A Laboratory Manual”, 1989, 2.sup.nd Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5.sup.th Ed., John Wiley & Sons: Secaucus, N.J.). For example, oligonucleotides may be prepared using any of a variety of chemical techniques well-known in the art, including, for example, chemical synthesis and polymerization based on a template as described, for example, in S. A. Narang et al., Meth. Enzymol. 1979, 68: 90-98; E. L. Brown et al., Meth. Enzymol. 1979, 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; and U.S. Pat. No. 4,458,066.

For example, oligonucleotides may be prepared using an automated, solid-phase procedure based on the phosphoramidite approach. In such a method, each nucleotide is individually added to the 5′-end of the growing oligonucleotide chain, which is attached at the 3′-end to a solid support. The added nucleotides are in the form of trivalent 3′-phosphoramidites that are protected from polymerization by a dimethoxytriyl (or DMT) group at the 5′-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on oligo synthesizers such as those commercially available from Perkin Elmer/Applied Biosystems, Inc. (Foster City, Calif.), DuPont (Wilmington, Del.) or Milligen (Bedford, Mass.). Alternatively, oligonucleotides can be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), and many others.

Purification of the oligonucleotides of the invention, where necessary or desirable, may be carried out by any of a variety of methods well-known in the art. Purification of oligonucleotides is typically performed either by native acrylamide gel electrophoresis, by anion-exchange HPLC as described, for example, by J. D. Pearson and F. E. Regnier (J. Chrom., 1983, 255: 137-149) or by reverse phase HPLC (G. D. McFarland and P. N. Borer, Nucleic Acids Res., 1979, 7: 1067-1080).

The sequence of oligonucleotides can be verified using any suitable sequencing method including, but not limited to, chemical degradation (A. M. Maxam and W. Gilbert, Methods of Enzymology, 1980, 65: 499-560), matrix-assisted laser desorption ionization time-of-flight (MALDI-TOF) mass spectrometry (U. Pieles et al., Nucleic Acids Res., 1993, 21: 3191-3196), mass spectrometry following a combination of alkaline phosphatase and exonuclease digestions (H. Wu and H. Aboleneen, Anal. Biochem., 2001, 290: 347-352), and the like.

As already mentioned above, modified oligonucleotides may be prepared using any of several means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc), or charged linkages (e.g., phosphorothioates, phosphorodithioates, etc). Oligonucleotides may contain one or more additional covalently linked moieties, such as, for example, proteins (e.g., nucleases, toxins, antibodies, signal peptides, poly-L-lysine, etc), intercalators (e.g., acridine, psoralen, etc), chelators (e.g., metals, radioactive metals, iron, oxidative metals, etc), and alkylators. The oligonucleotide may also be derivatized by formation of a methyl or ethyl phosphotriester or an alkyl phosphoramidate linkage. Furthermore, the oligonucleotide sequences of the present invention may also be modified with a label.

In certain embodiments, the detection probes or amplification primers or both probes and primers are labeled with a detectable agent or moiety before being used in amplification/detection assays. In certain embodiments, the detection probes are labeled with a detectable agent. Preferably, a detectable agent is selected such that it generates a signal which can be measured and whose intensity is related (e.g., proportional) to the amount of amplification products in the sample being analyzed.

The association between the oligonucleotide and detectable agent can be covalent or non-covalent. Labeled detection probes can be prepared by incorporation of or conjugation to a detectable moiety. Labels can be attached directly to the nucleic acid sequence or indirectly (e.g., through a linker). Linkers or spacer arms of various lengths are known in the art and are commercially available, and can be selected to reduce steric hindrance, or to confer other useful or desired properties to the resulting labeled molecules (see, for example, E. S. Mansfield et al., Mol. Cell. Probes, 1995, 9: 145-156).

Methods for labeling nucleic acid molecules are well-known in the art. For a review of labeling protocols, label detection techniques, and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachments of fluorescent dyes (L. M. Smith et al., Nucl. Acids Res., 1985, 13: 2399-2412) or of enzymes (B. A. Connoly and O. Rider, Nucl. Acids. Res., 1985, 13: 4485-4502); chemical modifications of nucleic acid molecules making them detectable immunochemically or by other affinity reactions (T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl. Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232). More recently developed nucleic acid labeling systems include, but are not limited to: ULS (Universal Linkage System), which is based on the reaction of mono-reactive cisplatin derivatives with the N7 position of guanine moieties in DNA (R. J. Heetebrij et al., Cytogenet. Cell. Genet. 1999, 87: 47-52), psoralen-biotin, which intercalates into nucleic acids and upon UV irradiation becomes covalently bonded to the nucleotide bases (C. Levenson et al., Methods Enzymol. 1990, 184: 577-583; and C. Pfannschmidt et al., Nucleic Acids Res. 1996, 24: 1702-1709), photoreactive azido derivatives (C. Neves et al., Bioconjugate Chem. 2000, 11: 51-55), and DNA alkylating agents (M. G. Sebestyen et al., Nat. Biotechnol. 1998, 16: 568-576).

Any of a wide variety of detectable agents can be used in the practice of the present invention. Suitable detectable agents include, but are not limited to, various ligands, radionuclides (such as, for example, ³²P, ³⁵S, ³H, ¹⁴C, ¹²⁵I, ¹³¹I, and the like); fluorescent dyes (for specific exemplary fluorescent dyes, see below); chemiluminescent agents (such as, for example, acridinium esters, stabilized dioxetanes, and the like); spectrally resolvable inorganic fluorescent semiconductor nanocrystals (i.e., quantum dots), metal nanoparticles (e.g., gold, silver, copper and platinum) or nanoclusters; enzymes (such as, for example, those used in an ELISA, i.e., horseradish peroxidase, beta-galactosidase, luciferase, alkaline phosphatase); colorimetric labels (such as, for example, dyes, colloidal gold, and the like); magnetic labels (such as, for example, Dynabeads™); and biotin, dioxigenin or other haptens and proteins for which antisera or monoclonal antibodies are available.

In certain embodiments, the detection probes are fluorescently labeled. Numerous known fluorescent labeling moieties of a wide variety of chemical structures and physical characteristics are suitable for use in the practice of this invention. Suitable fluorescent dyes include, but are not limited to, fluorescein and fluorescein dyes (e.g., fluorescein isothiocyanine or FITC, naphthofluorescein, 4′,5′-dichloro-2′,7′-dimethoxy-fluorescein, 6 carboxyfluorescein or FAM), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red.™., Spectrum Green.™., cyanine dyes (e.g., Cy-3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. For more examples of suitable fluorescent dyes and methods for linking or incorporating fluorescent dyes to nucleic acid molecules see, for example, “The Handbook of Fluorescent Probes and Research Products”, 9th Ed., Molecular Probes, Inc., Eugene, Oreg. Fluorescent dyes as well as labeling kits are commercially available from, for example, Amersham Biosciences, Inc. (Piscataway, N.J.), Molecular Probes Inc. (Eugene, Oreg.), and New England Biolabs Inc. (Berverly, Mass.).

As mentioned, identification of the RNA marker may be carried out using an amplification reaction.

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR) and ligase chain reaction (LCR). In a typical PCR amplification reaction, a nucleic acid sequence of interest is often amplified at least fifty thousand fold in amount over its amount in the starting sample. A “copy” or “amplicon” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification.

A typical amplification reaction is carried out by contacting a forward and reverse primer (a primer pair) to the sample DNA together with any additional amplification reaction reagents under conditions which allow amplification of the target sequence.

The terms “forward primer” and “forward amplification primer” are used herein interchangeably, and refer to a primer that hybridizes (or anneals) to the target (template strand). The terms “reverse primer” and “reverse amplification primer” are used herein interchangeably, and refer to a primer that hybridizes (or anneals) to the complementary target strand. The forward primer hybridizes with the target sequence 5′ with respect to the reverse primer.

The term “amplification conditions”, as used herein, refers to conditions that promote annealing and/or extension of primer sequences. Such conditions are well-known in the art and depend on the amplification method selected. Thus, for example, in a PCR reaction, amplification conditions generally comprise thermal cycling, i.e., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and temperature cycling, buffer, salt, ionic strength, and pH, and the like.

As used herein, the term “amplification reaction reagents”, refers to reagents used in nucleic acid amplification reactions and may include, but are not limited to, buffers, reagents, enzymes having reverse transcriptase and/or polymerase activity or exonuclease activity, enzyme cofactors such as magnesium or manganese, salts, nicotinamide adenine dinuclease (NAD) and deoxynucleoside triphosphates (dNTPs), such as deoxyadenosine triphospate, deoxyguanosine triphosphate, deoxycytidine triphosphate and thymidine triphosphate. Amplification reaction reagents may readily be selected by one skilled in the art depending on the amplification method used.

According to this aspect of the present invention, the amplifying may be effected using techniques such as polymerase chain reaction (PCR), which includes, but is not limited to Allele-specific PCR, Assembly PCR or Polymerase Cycling Assembly (PCA), Asymmetric PCR, Helicase-dependent amplification, Hot-start PCR, Intersequence-specific PCR (ISSR), Inverse PCR, Ligation-mediated PCR, Methylation-specific PCR (MSP), Miniprimer PCR, Multiplex Ligation-dependent Probe Amplification, Multiplex-PCR, Nested PCR, Overlap-extension PCR, Quantitative PCR (Q-PCR), Reverse Transcription PCR (RT-PCR), Solid Phase PCR: encompasses multiple meanings, including Polony Amplification (where PCR colonies are derived in a gel matrix, for example), Bridge PCR (primers are covalently linked to a solid-support surface), conventional Solid Phase PCR (where Asymmetric PCR is applied in the presence of solid support bearing primer with sequence matching one of the aqueous primers) and Enhanced Solid Phase PCR (where conventional Solid Phase PCR can be improved by employing high Tm and nested solid support primer with optional application of a thermal ‘step’ to favour solid support priming), Thermal asymmetric interlaced PCR (TAIL-PCR), Touchdown PCR (Step-down PCR), PAN-AC and Universal Fast Walking.

The PCR (or polymerase chain reaction) technique is well-known in the art and has been disclosed, for example, in K. B. Mullis and F. A. Faloona, Methods Enzymol., 1987, 155: 350-355 and U.S. Pat. Nos. 4,683,202; 4,683,195; and 4,800,159 (each of which is incorporated herein by reference in its entirety). In its simplest form, PCR is an in vitro method for the enzymatic synthesis of specific DNA sequences, using two oligonucleotide primers that hybridize to opposite strands and flank the region of interest in the target DNA. A plurality of reaction cycles, each cycle comprising: a denaturation step, an annealing step, and a polymerization step, results in the exponential accumulation of a specific DNA fragment (“PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; R. K. Saiki et al., Nature, 1986, 324: 163-166). The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). RNA target sequences may be amplified by reverse transcribing the mRNA into cDNA, and then performing PCR (RT-PCR), as described above. Alternatively, a single enzyme may be used for both steps as described in U.S. Pat. No. 5,322,770.

The duration and temperature of each step of a PCR cycle, as well as the number of cycles, are generally adjusted according to the stringency requirements in effect. Annealing temperature and timing are determined both by the efficiency with which a primer is expected to anneal to a template and the degree of mismatch that is to be tolerated. The ability to optimize the reaction cycle conditions is well within the knowledge of one of ordinary skill in the art. Although the number of reaction cycles may vary depending on the detection analysis being performed, it usually is at least 15, more usually at least 20, and may be as high as 60 or higher. However, in many situations, the number of reaction cycles typically ranges from about 20 to about 40.

The denaturation step of a PCR cycle generally comprises heating the reaction mixture to an elevated temperature and maintaining the mixture at the elevated temperature for a period of time sufficient for any double-stranded or hybridized nucleic acid present in the reaction mixture to dissociate. For denaturation, the temperature of the reaction mixture is usually raised to, and maintained at, a temperature ranging from about 85° C. to about 100° C., usually from about 90° C. to about 98° C., and more usually from about 93° C. to about 96° C., for a period of time ranging from about 3 to about 120 seconds, usually from about 5 to about 30 seconds.

Following denaturation, the reaction mixture is subjected to conditions sufficient for primer annealing to template DNA present in the mixture. The temperature to which the reaction mixture is lowered to achieve these conditions is usually chosen to provide optimal efficiency and specificity, and generally ranges from about 50° C. to about ° C., usually from about 55° C., to about 70° C., and more usually from about 60° C. to about 68° C. Annealing conditions are generally maintained for a period of time ranging from about 15 seconds to about 30 minutes, usually from about 30 seconds to about 5 minutes.

Following annealing of primer to template DNA or during annealing of primer to template DNA, the reaction mixture is subjected to conditions sufficient to provide for polymerization of nucleotides to the primer's end in a such manner that the primer is extended in a 5′ to 3′ direction using the DNA to which it is hybridized as a template, (i.e., conditions sufficient for enzymatic production of primer extension product). To achieve primer extension conditions, the temperature of the reaction mixture is typically raised to a temperature ranging from about 65° C. to about 75° C., usually from about 67° C. to about 73° C., and maintained at that temperature for a period of time ranging from about 15 seconds to about 20 minutes, usually from about 30 seconds to about 5 minutes.

The above cycles of denaturation, annealing, and polymerization may be performed using an automated device typically known as a thermal cycler or thermocycler. Thermal cyclers that may be employed are described in U.S. Pat. Nos. 5,612,473; 5,602,756; 5,538,871; and 5,475,610 (each of which is incorporated herein by reference in its entirety). Thermal cyclers are commercially available, for example, from Perkin Elmer-Applied Biosystems (Norwalk, Conn.), BioRad (Hercules, Calif.), Roche Applied Science (Indianapolis, Ind.), and Stratagene (La Jolla, Calif.).

Amplification products obtained using primers of the present invention may be detected using agarose gel electrophoresis and visualization by ethidium bromide staining and exposure to ultraviolet (UV) light or by sequence analysis of the amplification product.

According to one embodiment, the amplification and quantification of the amplification product may be effected in real-time (qRT-PCR). Typically, QRT-PCR methods use double stranded DNA detecting molecules to measure the amount of amplified product in real time.

As used herein the phrase “double stranded DNA detecting molecule” refers to a double stranded DNA interacting molecule that produces a quantifiable signal (e.g., fluorescent signal). For example such a double stranded DNA detecting molecule can be a fluorescent dye that (1) interacts with a fragment of DNA or an amplicon and (2) emits at a different wavelength in the presence of an amplicon in duplex formation than in the presence of the amplicon in separation. A double stranded DNA detecting molecule can be a double stranded DNA intercalating detecting molecule or a primer-based double stranded DNA detecting molecule.

A double stranded DNA intercalating detecting molecule is not covalently linked to a primer, an amplicon or a nucleic acid template. The detecting molecule increases its emission in the presence of double stranded DNA and decreases its emission when duplex DNA unwinds. Examples include, but are not limited to, ethidium bromide, YO-PRO-1, Hoechst 33258, SYBR Gold, and SYBR Green I. Ethidium bromide is a fluorescent chemical that intercalates between base pairs in a double stranded DNA fragment and is commonly used to detect DNA following gel electrophoresis. When excited by ultraviolet light between 254 nm and 366 nm, it emits fluorescent light at 590 nm. The DNA-ethidium bromide complex produces about 50 times more fluorescence than ethidium bromide in the presence of single stranded DNA. SYBR Green I is excited at 497 nm and emits at 520 nm. The fluorescence intensity of SYBR Green I increases over 100 fold upon binding to double stranded DNA against single stranded DNA. An alternative to SYBR Green I is SYBR Gold introduced by Molecular Probes Inc. Similar to SYBR Green I, the fluorescence emission of SYBR Gold enhances in the presence of DNA in duplex and decreases when double stranded DNA unwinds. However, SYBR Gold's excitation peak is at 495 nm and the emission peak is at 537 nm. SYBR Gold reportedly appears more stable than SYBR Green I. Hoechst 33258 is a known bisbenzimide double stranded DNA detecting molecule that binds to the AT rich regions of DNA in duplex. Hoechst 33258 excites at 350 nm and emits at 450 nm. YO-PRO-1, exciting at 450 nm and emitting at 550 nm, has been reported to be a double stranded DNA specific detecting molecule. In a particular embodiment of the present invention, the double stranded DNA detecting molecule is SYBR Green I.

A primer-based double stranded DNA detecting molecule is covalently linked to a primer and either increases or decreases fluorescence emission when amplicons form a duplex structure. Increased fluorescence emission is observed when a primer-based double stranded DNA detecting molecule is attached close to the 3′ end of a primer and the primer terminal base is either dG or dC. The detecting molecule is quenched in the proximity of terminal dC-dG and dG-dC base pairs and dequenched as a result of duplex formation of the amplicon when the detecting molecule is located internally at least 6 nucleotides away from the ends of the primer. The dequenching results in a substantial increase in fluorescence emission. Examples of these type of detecting molecules include but are not limited to fluorescein (exciting at 488 nm and emitting at 530 nm), FAM (exciting at 494 nm and emitting at 518 nm), JOE (exciting at 527 and emitting at 548), HEX (exciting at 535 nm and emitting at 556 nm), TET (exciting at 521 nm and emitting at 536 nm), Alexa Fluor 594 (exciting at 590 nm and emitting at 615 nm), ROX (exciting at 575 nm and emitting at 602 nm), and TAMRA (exciting at 555 nm and emitting at 580 nm). In contrast, some primer-based double stranded DNA detecting molecules decrease their emission in the presence of double stranded DNA against single stranded DNA. Examples include, but are not limited to, rhodamine, and BODIPY-FI (exciting at 504 nm and emitting at 513 nm). These detecting molecules are usually covalently conjugated to a primer at the 5′ terminal dC or dG and emit less fluorescence when amplicons are in duplex. It is believed that the decrease of fluorescence upon the formation of duplex is due to the quenching of guanosine in the complementary strand in close proximity to the detecting molecule or the quenching of the terminal dC-dG base pairs.

According to one embodiment, the primer-based double stranded DNA detecting molecule is a 5′ nuclease probe. Such probes incorporate a fluorescent reporter molecule at either the 5′ or 3′ end of an oligonucleotide and a quencher at the opposite end. The first step of the amplification process involves heating to denature the double stranded DNA target molecule into a single stranded DNA. During the second step, a forward primer anneals to the target strand of the DNA and is extended by Taq polymerase. A reverse primer and a 5′ nuclease probe then anneal to this newly replicated strand.

In this embodiment, at least one of the primer pairs or 5′ nuclease probe should hybridize with a unique determinant sequence. The polymerase extends and cleaves the probe from the target strand. Upon cleavage, the reporter is no longer quenched by its proximity to the quencher and fluorescence is released. Each replication will result in the cleavage of a probe. As a result, the fluorescent signal will increase proportionally to the amount of amplification product.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Study Description

The study included 54 patients (49 infectious and 5 healthy), out of which 19 were females (35%) and 35 were males (65%). The mean age of the patients was 34 years old, ranging from 5 months to 88 years old. Patients exhibited a variety of clinical syndromes, infectious etiology and disease outcomes. All patients had a serum blood measurement taken during their hospitalization or emergency department visit course. Detailed characterization of patients included in the study is described in Table 1, herein below.

TABLE 1

| | | | | Children | | Adults | |
|---|---|---|---|---|---|---|---|
| | | Total | | (≤18 years) | | (>18 years) | |
| Criteria | | n = 54 | | n = 22 | | n = 32 | |
| Age (Years) | | | | | | | |
| | <3 | 10 | (19%) | | | | |
| | 3-6 | 8 | (15%) | | | | |
| | 6-9 | 2 | (4%) | | | | |
| | 9-18 | 2 | (4%) | | | | |
| | 18-30 | 4 | (7%) | | | | |
| | 30-50 | 9 | (17%) | | | | |
| | 50-70 | 12 | (22%) | | | | |
| | 70-90 | 8 | (15%) | | | | |
| | >90 | — | | | | | |
| Gender | | | | | | | |
| | Female | 19 | (35%) | 4 | (18%) | 15 | (47%) |
| | Male | 35 | (65%) | 18 | (82%) | 17 | (53%) |
| Maximal | | | | | | | |
| Temperature | | | | | | | |
| (° C.) | | | | | | | |
| | <37.5 | — | | | | | |
| | 37.5-38.4 | 10 | (20%) | 3 | (14%) | 7 | (26%) |
| | 38.5-39.4 | 29 | (59%) | 11 | (50%) | 18 | (67%) |
| | 39.5-40.4 | 8 | (16%) | 6 | (27%) | 2 | (7%) |
| | >40.5 | 2 | (4%) | 2 | (9%) | — | |
| Etiology | | | | | | | |
| | Bacterial | 32 | (59%) | 12 | (55%) | 20 | (63%) |
| | Viral | 17 | (32%) | 10 | (46%) | 7 | (22%) |
| | Healthy | 5 | (9%) | — | | 5 | (16%) |
| Clinical | | | | | | | |
| Syndrome | | | | | | | |
| LRTI | | 16 | (33%) | 9 | (41%) | 7 | (26%) |
| URTI | | 11 | (22%) | 7 | (32%) | 4 | (15%) |
| Unspecified | | 5 | (10%) | 3 | (14%) | 2 | (7%) |
| viral | | | | | | | |
| infection | | | | | | | |
| Pyelonephritis/ | | 4 | (8%) | 2 | (9%) | 2 | (7%) |
| UTI | | | | | | | |
| Intra- | | 2 | (4%) | — | | 2 | (7%) |
| abdominal | | | | | | | |
| infection | | | | | | | |
| Other | | 11 | (22%) | 1 | (5%) | 10 | (37%) |
| Time from | | | | | | | |
| symptoms | | | | | | | |
| onset | | | | | | | |
| (days) | | | | | | | |
| | 0-2 | 28 | (57%) | 14 | (64%) | 14 | (52%) |
| | ≥3 | 21 | (43%) | 8 | (36%) | 13 | (48%) |
| Hospitalization | | | | | | | |
| duration | | | | | | | |
| (days) | | | | | | | |
| | Not | 7 | (14%) | 2 | (9%) | 5 | (19%) |
| | hospitalized | | | | | | |
| | 1-2 | 12 | (25%) | 7 | (32%) | 5 | (19%) |
| | ≥3 | 29 | (59%) | 13 | (59%) | 16 | (59%) |
| Antibiotic | | | | | | | |
| prescription | | | | | | | |
| | Yes | 39 | (80%) | 15 | (68%) | 24 | (89%) |
| | No | 10 | (20%) | 7 | (32%) | 3 | (11%) |

* Values are presented as total numbers, followed by the corresponding percentages in brackets. The percentages in the following criteria are calculated out of a total of patients not including the healthy subjects: maximal temperature, clinical syndrome, time from symptoms onset, hospitalization duration, antibiotic prescription. LRTI (lower respiratory tract infection) includes the following clinical syndromes: LRTI, Asthma Exacerbation, Acute Bronchitis, Pneumonia, Bronchiolitis. URTI (upper respiratory tract infection) includes the following clinical syndromes: UTRI, Acute Tonsillitis, OME (Otitis Media with Effusion). Unspecified viral infection category includes Influenza and other unspecified viral infections. Intra-abdominal infection category includes Gastroenteritis, Peritonitis. The following clinical syndromes are classified as ‘Other’: Septic shock, Bacteremia, Mastitis, Meningitis, Cellulitis, Mediastinitis.

Patients were categorized into two distinct cohorts: etiology cohort (according to the underlying etiology: viral or bacterial) and disease severity cohort (according to the disease severity status: severe or non severe).

Etiology cohort: A total of 27 infectious patients were categorized as having either a viral or bacterial infectious etiology: 13 had a bacterial infection and 14 had a viral infection. Infectious etiology was established by applying a rigorous expert panel adjudication process.

Disease severity cohort: A total of 24 infectious patients were categorized as having either a severe or non-severe disease outcome: 12 severe and 12 non-severe. A severe disease outcome was defined for patients meeting either one of the following criteria:
ICU admission;
Mechanical ventilation; or
28-day mortality

A non-severe label was given to patients not meeting any one of the upper mentioned severity criteria. Two patients of the disease severity cohort are also included in the etiology cohort (i.e., the disease severity cohort adds 22 new patients).

The study main goal was to identify novel biomarkers for disease severity and for differentiating between bacterial and viral infections. An additional, cross cohorts', analysis was made in order to identify viral and bacterial markers that are unaffected by disease severity, and vice versa.

Materials and Methods

Protein screening was performed using Olink Proteomics' PEA technology. In total, 184 proteins from two different panels were measured (Olink® Target 96 Inflammation (v.3022), Olink® Target 96 Immune Response (v.3203)). The resulting protein measurements enable relative quantification, where the results are expressed as normalized protein expression (NPX), arbitrary units on a log2-scale.

RNA screening was performed using meta-analysis of published gene expression studies. Comparison of severe infection to non-severe infection was done based on 4 studies of viral infection, totalling 97 patients with severe infection and 184 patients with non-severe infection. Comparison of bacterial infection to viral infection was done based on 8 studies (not overlapping with the 4 studies of infection severity) totalling 306 patients with a bacterial infection and 392 patients with a viral infection. Genes included in the severity meta-analysis were measured in at least 2 of the 4 severity studies. Genes included in the B-V meta-analysis were measured in at least 4 of the 8 B-V studies. In total, 16,939 genes were included in both meta-analyses and in the evaluation of the interaction between infection etiology and infection severity.

Measurement of Biomarker Accuracy

The level of expression of each protein along the different samples and sub-groups was compared by calculating the delta between the medians of the protein in the two comparison groups. Since the measurements are reported in NPX arbitrary units on a log 2-scale, the linear fold-change of each protein is calculated as 2^{delta of medians (NPX)}. P-value is calculated using Mann-Whitney U test. Only proteins with a p-value of <0.05 were considered. Bidirectional biomarkers are proteins that exhibit different directionality between the two comparison groups (e.g., are up-regulated in bacterial infections and down-regulated in viral infections). Specific biomarkers for bacterial/viral infections were defined as proteins that significantly differentiate bacterial/viral infections (P-value <0.05) and are non-significant in differentiating severe/non-severe infections (P-value ≥0.05), and vice versa for specific biomarkers for severe/non-severe infections (see Tables 3, 5 accordingly).

Results

Protein biomarkers marked in bold in the following tables are the top markers with ROC AUC >0.9 and the highest absolute linear fold-change.

CXCL11 was found to be a specific viral biomarker, MCP-2—specific viral biomarker, OSM—specific bacterial biomarker, IL6—up-regulated in bacterial infections, HGF—up-regulated in bacterial infections, TNFB—up-regulated in viral infections and down-regulated in bacterial infections, TREM1+up-regulated in bacterial infections and down-regulated in viral infections, CLEC4D—up-regulated in bacterial infections and down-regulated in viral infections, TRAIL—up-regulated in viral infections and down-regulated in bacterial infections, HSD11B1—specific viral biomarker, LAMPS—specific viral biomarker and LAGS—specific viral biomarker. IL-8—specific disease severity biomarker, CKAP4—up-regulated in severe infections, MCP-3—up-regulated in severe infections, AREG—specific disease severity biomarker, PSIP1—specific disease severity biomarker, EN-RAGE—up-regulated in severe infections and down-regulated in non-severe infections, LILRB4—specific disease severity biomarker, KRT19—specific disease severity biomarker, TWEAK—specific disease severity biomarker, CLEC7A—specific disease severity biomarker, IL-18R1—specific disease severity biomarker and DCBLD2—specific disease severity biomarker.

Table 2 summarizes the statistically significant protein biomarkers differentiating between bacterial and viral infections. (Biomarkers marked in bold in the following tables are the top markers with ROC AUC >0.9 and the highest absolute linear fold-change).

TABLE 2
Bacterial Protein Biomarkers

| Up-regulated in bacterial | | Down-regulated in bacterial | | Up-regulated in bacterial, | |
|---|---|---|---|---|---|
| infections | | infections | | down in viral | |
| UniProt ID | Biomarker | UniProt ID | Biomarker | UniProt ID | Biomarker |
| Q6UXB4 | CLEC4G | P51671 | CCL11 | Q9GZT9 | EGLN1 |
| P05231 | IL6 | P58499 | FAM3B | Q8WXI8 | CLEC4D |
| Q07065 | CKAP4 | Q99616 | MCP-4 | Q04759 | PRKCQ |
| Q05084 | ICA1 | O15444 | CCL25 | Q9NP99 | TREM1 |
| Q9Y2J8 | PADI2 | | | P01135 | TGF-alpha |
| P15692 | VEGFA | | | P42830 | CXCL5 |
| P13725 | OSM | | | P55773 | CCL23 |
| O43557 | TNFSF14 | | | P80511 | EN-RAGE |
| P14210 | HGF | | | | |
| Q13007 | IL-24 | | | | |
| P15018 | LIF | | | | |

Viral Protein Biomarkers

| Up-regulated in viral | | Down-regulated in viral | | Up-regulated in viral, down | |
|---|---|---|---|---|---|
| infections | | infections | | in bacterial | |
| UniProt ID | Biomarker | UniProt ID | Biomarker | UniProt ID | Biomarker |
| P08727 | KRT19 | P01138 | Beta-NGF | Q8WTT0 | CLEC4C |
| O60449 | LY75 | | | P28845 | HSD11B1 |
| Q6DN72 | FCRL6 | | | P18627 | LAG3 |
| Q9UQV4 | LAMP3 | | | P50591 | TRAIL |
| P01732 | CD8A | | | P21583 | SCF |
| P80098 | MCP-3 | | | Q8N6P7 | IL-22 RA1 |
| O14625 | CXCL11 | | | P49771 | Flt3L |
| Q07325 | CXCL9 | | | P80075 | MCP-2 |
| Q9NZQ7 | PD-L1 | | | P01374 | TNFB |
| P29460 | IL-12B | | | | |
| P01375 | TNF | | | | |
| P01579 | IFN-gamma | | | | |
| P78423 | CX3CL1 | | | | |

TABLE 3
Statistically significant specific protein biomarkers for bacterial infections
Specific protein biomarkers for bacterial infections

| UniProt ID | Biomarker |
|---|---|
| Q6UXB4 | CLEC4G |
| P51671 | CCL11 |
| Q04759 | PRKCQ |
| P58499 | FAM3B |
| P15692 | VEGFA |
| P13725 | OSM |
| P01135 | TGF-alpha |
| Q99616 | MCP-4 |
| O43557 | TNFSF14 |
| P42830 | CXCL5 |
| O15444 | CCL25 |

TABLE 4
Statistically significant specific protein biomarkers for viral infections
Specific protein biomarkers for viral infections

| UniProt ID | Biomarker |
|---|---|
| O60449 | LY75 |
| P28845 | HSD11B1 |
| Q6DN72 | FCRL6 |
| Q9UQV4 | LAMP3 |
| P18627 | LAG3 |
| O14625 | CXCL11 |
| Q07325 | CXCL9 |
| Q8N6P7 | IL-22 RA1 |
| P01138 | Beta-NGF |
| P01375 | TNF |
| P49771 | Flt3L |
| P01579 | IFN-gamma |
| P80075 | MCP-2 |
| P78423 | CX3CL1 |

Table 5 summarizes the statistically significant protein biomarkers differentiating between severe and non-severe infections. (Biomarkers marked in bold in the following tables are the top markers with ROC AUC >0.9 and the highest absolute linear fold-change).

TABLE 5
Severe protein Biomarkers

| Up-regulated in severe | | Down-regulated in | | Up-regulated in severe, | |
|---|---|---|---|---|---|
| infections | | severe infections | | down in non-severe | |
| UniProt ID | Biomarker | UniProt ID | Biomarker | UniProt ID | Biomarker |
| Q05516 | ZBTB16 | Q8WTT0 | CLEC4C | O75475 | PSIP1 |
| P05231 | IL6 | Q9UMR7 | CLEC4A | P14317 | HCLS1 |
| Q06830 | PRDX1 | Q9UKX5 | ITGA11 | P30044 | PRDX5 |
| P30048 | PRDX3 | P50591 | TRAIL | Q9C035 | TRIM5 |
| Q8NHJ6 | LILRB4 | P14784 | IL-2RB | Q9GZT9 | EGLN1 |
| P08727 | KRT19 | P21583 | SCF | P78362 | SRPK2 |
| P50135 | HNMT | Q8NFT8 | DNER | O14867 | BACH1 |
| Q07065 | CKAP4 | O43508 | TWEAK | Q05084 | ICA1 |
| O94992 | HEXIM1 | P01374 | TNFB | Q96PD2 | DCBLD2 |
| Q8WXI8 | CLEC4D | | | Q9BXN2 | CLEC7A |
| Q9NP99 | TREM1 | | | P80511 | EN-RAGE |
| P78310 | CXADR | | | P25942 | CD40 |
| P22301 | IL10 | | | | |
| P52823 | STC1 | | | | |
| O00273 | DFFA | | | | |
| P15514 | AREG | | | | |
| Q9Y2J8 | PADI2 | | | | |
| P78410 | BTN3A2 | | | | |
| P10145 | IL8 | | | | |
| P80098 | MCP-3 | | | | |
| O00300 | OPG | | | | |
| Q14116 | IL18 | | | | |
| Q9GZV9 | FGF-23 | | | | |
| Q13261 | IL-15RA | | | | |
| Q13478 | IL-18R1 | | | | |
| Q9NZQ7 | PD-L1 | | | | |
| P14210 | HGF | | | | |
| Q13007 | IL-24 | | | | |
| P55773 | CCL23 | | | | |
| P10147 | CCL3 | | | | |
| P15018 | LIF | | | | |
| P78556 | CCL20 | | | | |

Non-Severe protein Biomarkers

| Up-regulated in non- | | Down-regulated in non- | |
|---|---|---|---|
| severe, down in severe | | severe infections | |
| UniProt ID | Biomarker | UniProt ID | Biomarker |
| P16278 | GLB1 | P01732 | CD8A |
| | | Q9BZW8 | CD244 |
| | | P29460 | IL-12B |

TABLE 6
Statistically significant specific protein biomarkers for severe infections
Specific protein biomarkers for severe infections

| UniProt ID | Biomarker |
|---|---|
| O75475 | PSIP1 |
| Q05516 | ZBTB16 |
| P14317 | HCLS1 |
| Q9UMR7 | CLEC4A |
| Q06830 | PRDX1 |
| P30048 | PRDX3 |
| P30044 | PRDX5 |
| Q9C035 | TRIM5 |
| Q8NHJ6 | LILRB4 |
| P50135 | HNMT |
| O94992 | HEXIM1 |
| P78310 | CXADR |
| P22301 | IL10 |
| P78362 | SRPK2 |
| O14867 | BACH1 |
| P52823 | STC1 |
| O00273 | DFFA |
| Q96PD2 | DCBLD2 |
| P15514 | AREG |
| Q9BXN2 | CLEC7A |
| Q9UKX5 | ITGA11 |
| P78410 | BTN3A2 |
| P10145 | IL8 |
| O00300 | OPG |
| P14784 | IL-2RB |
| Q14116 | IL18 |
| Q9GZV9 | FGF-23 |
| Q13261 | IL-15RA |
| Q13478 | IL-18R1 |
| P10147 | CCL3 |
| Q8NFT8 | DNER |
| P25942 | CD40 |
| O43508 | TWEAK |
| P78556 | CCL20 |

TABLE 7
Statistically significant specific protein
biomarkers for non-severe infections
Specific biomarkers for non-severe infections

| UniProt ID | Biomarker |
|---|---|
| Q9BZW8 | CD244 |
| P16278 | GLB1 |

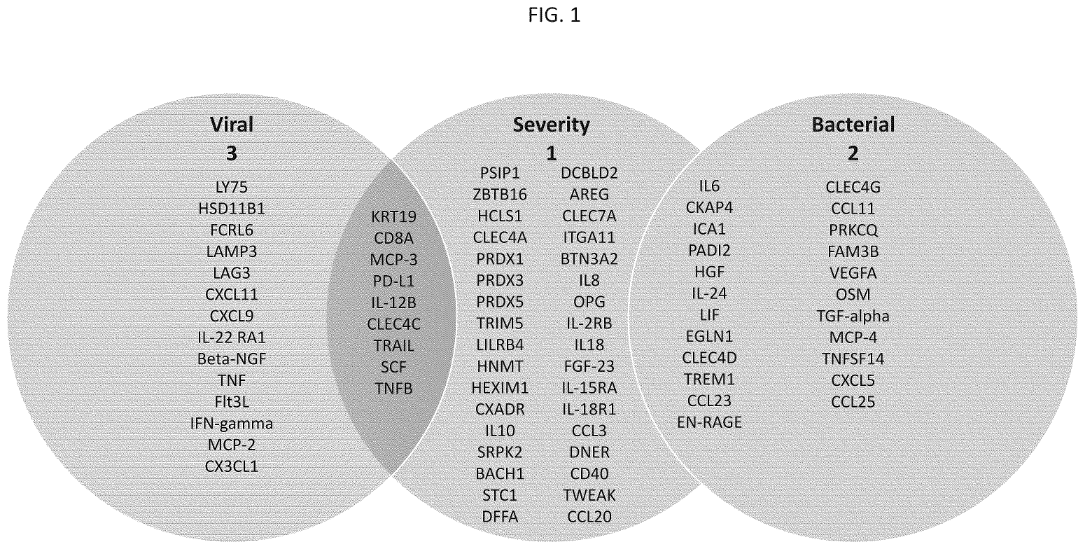

The results summarized in Tables 2-6 are displayed graphically in FIG. 1.

RNA markers whose expression is elevated during a severe infection but does not distinguish between a bacterial and viral infection are summarized in Table 8 (severity AUROC >0.8, BV AUROC <0.7).

TABLE 8

| | RefSeq | Severity | ROC | B-V | ROC |
|---|---|---|---|---|---|
| | mRNA | delta | AUC | delta | AUC |
| ELANE | NM_001972.4 | 2.69 | 0.82 | 0.92 | 0.60 |
| CEACAM8 | NM_001816.4 | 2.64 | 0.80 | 1.09 | 0.60 |
| MPO | NM_000250.2 | 2.58 | 0.83 | 0.69 | 0.61 |
| OLFM4 | NM_006418.5 | 2.54 | 0.81 | 1.26 | 0.64 |
| HP | NM_005143.5 | 2.29 | 0.88 | 1.00 | 0.67 |
| CEACAM6 | NM_002483.7 | 2.17 | 0.83 | 0.57 | 0.58 |
| ARG1 | NM_001244438.2 | 2.01 | 0.83 | 1.14 | 0.68 |
| DEFA1B | NM_001302265.2 | 1.98 | 0.81 | 0.80 | 0.60 |
| PRTN3 | NM_002777.4 | 1.90 | 0.84 | 0.68 | 0.63 |
| S100A12 | NM_005621.2 | 1.53 | 0.84 | 0.19 | 0.69 |
| HPR | NM_001384360.1 | 1.43 | 0.81 | 0.11 | 0.57 |
| BMX | NM_203281.3 | 1.15 | 0.86 | 0.21 | 0.64 |
| GYG1 | NM_004130.4 | 1.12 | 0.86 | 0.51 | 0.66 |
| GADD45A | NM_001924.4 | 1.11 | 0.81 | 0.44 | 0.62 |
| UGCG | NM_003358.3 | 1.06 | 0.83 | 0.29 | 0.57 |
| BCL6 | NM_001130845.2 | 1.00 | 0.84 | 0.45 | 0.65 |
| GGH | NM_003878.3 | 0.99 | 0.81 | 0.14 | 0.51 |
| PYGL | NM_002863.5 | 0.95 | 0.82 | 0.07 | 0.68 |
| UPP1 | NM_001362774.2 | 0.92 | 0.82 | 0.24 | 0.70 |
| UPB1 | NM_016327.3 | 0.89 | 0.84 | 0.32 | 0.67 |
| CR1L | NM_175710.2 | 0.86 | 0.81 | −0.05 | 0.59 |
| BEX1 | NM_018476.4 | 0.85 | 0.81 | 0.22 | 0.61 |
| UBE2C | NM_007019.4 | 0.85 | 0.81 | −0.08 | 0.58 |
| PRC1 | NM_003981.4 | 0.84 | 0.81 | −0.02 | 0.56 |
| EXOSC4 | NM_019037.3 | 0.84 | 0.84 | 0.32 | 0.63 |
| CHIT1 | NM_003465.3 | 0.83 | 0.83 | 0.29 | 0.58 |
| HIST1H2BI | NM_138720.2 | 0.82 | 0.80 | 0.03 | 0.52 |
| PDZD8 | NM_173791.5 | 0.79 | 0.83 | 0.34 | 0.70 |
| SRPK1 | NM_003137.5 | 0.77 | 0.80 | 0.22 | 0.62 |
| TXN | NM_003329.4 | 0.76 | 0.86 | −0.04 | 0.63 |
| CEP55 | NM_018131.5 | 0.75 | 0.81 | −0.14 | 0.56 |
| DACH1 | NM_080759.6 | 0.73 | 0.83 | 0.31 | 0.68 |
| CD63 | NM_001780.6 | 0.71 | 0.82 | 0.39 | 0.68 |
| FLOT1 | NM_005803.4 | 0.67 | 0.83 | 0.48 | 0.70 |
| ABCB6 | NM_005689.4 | 0.67 | 0.82 | −0.09 | 0.53 |
| HSD3B7 | NM_025193.4 | 0.66 | 0.80 | 0.29 | 0.70 |
| DHCR7 | NM_001360.3 | 0.66 | 0.86 | 0.24 | 0.67 |
| MAFG | NM_032711.4 | 0.64 | 0.91 | 0.11 | 0.58 |
| SERPINB10 | NM_005024.3 | 0.64 | 0.81 | 0.32 | 0.67 |
| METTL9 | NM_016025.5 | 0.63 | 0.83 | 0.26 | 0.65 |
| HIST1H2AI | NM_003509.3 | 0.62 | 0.80 | −0.01 | 0.51 |
| TPST2 | NM_001362923.2 | 0.62 | 0.81 | 0.17 | 0.60 |
| KIF1B | NM_001365951.3 | 0.61 | 0.83 | 0.10 | 0.59 |
| DRAM1 | NM_018370.3 | 0.61 | 0.80 | 0.37 | 0.66 |
| WIPI1 | NM_017983.7 | 0.61 | 0.80 | 0.39 | 0.68 |
| ANO10 | NM_018075.5 | 0.60 | 0.82 | 0.37 | 0.69 |
| TP53I11 | NM_001258320.2 | 0.58 | 0.81 | 0.20 | 0.61 |
| LDHA | NM_005566.4 | 0.58 | 0.83 | 0.04 | 0.64 |
| NARF | NM_012336.4 | 0.57 | 0.82 | 0.40 | 0.70 |
| F12 | NM_000505.4 | 0.56 | 0.84 | 0.18 | 0.62 |
| LRPAP1 | NM_002337.4 | 0.56 | 0.80 | 0.31 | 0.61 |
| RILPL1 | NM_178314.5 | 0.55 | 0.80 | −0.04 | 0.52 |
| ZNF788 | | 0.52 | 0.81 | 0.12 | 0.51 |
| RAB27A | NM_004580.5 | 0.50 | 0.80 | 0.27 | 0.69 |
| MSRB3 | NM_198080.4 | 0.49 | 0.82 | 0.14 | 0.67 |
| PCMT1 | NM_005389.2 | 0.49 | 0.87 | −0.63 | 0.54 |
| STBD1 | NM_003943.5 | 0.49 | 0.84 | 0.17 | 0.60 |
| FAM89A | NM_198552.3 | 0.48 | 0.81 | 0.34 | 0.64 |
| HIST1H3G | NM_003534.3 | 0.46 | 0.83 | 0.06 | 0.57 |
| ENTPD7 | NM_001349962.2 | 0.45 | 0.85 | 0.27 | 0.68 |
| MEF2B | NM_001145785.2 | 0.45 | 0.82 | −0.03 | 0.53 |
| HGF | NM_000601.6 | 0.44 | 0.81 | 0.20 | 0.67 |
| SLC44A1 | NM_080546.5 | 0.41 | 0.82 | −0.02 | 0.52 |
| PPP4R2 | NM_174907.4 | 0.40 | 0.84 | −0.03 | 0.54 |
| HSD17B12 | NM_016142.3 | 0.37 | 0.83 | −0.19 | 0.55 |
| KCNK5 | NM_003740.4 | 0.37 | 0.81 | 0.07 | 0.55 |
| HMGB3 | NM_001301228.2 | 0.36 | 0.81 | 0.15 | 0.65 |
| PDE6H | NM_006205.3 | 0.35 | 0.81 | 0.23 | 0.66 |
| PIR | NM_003662.4 | 0.35 | 0.83 | 0.01 | 0.52 |
| DCTN2 | NM_006400.5 | 0.25 | 0.80 | 0.14 | 0.63 |
| TMED8 | NM_001346131.2 | 0.23 | 0.82 | 0.09 | 0.64 |
| TCTEX1D1 | NM_152665.3 | 0.21 | 0.81 | 0.18 | 0.67 |

RNA markers whose expression is decreased during a severe infection but does not distinguish between a bacterial and viral infection are summarized in Table 9 severity AUROC >0.8, BV AUROC <0.7).

TABLE 9

| | | | | | B-V |
|---|---|---|---|---|---|
| | RefSeq | Severity | ROC | | ROC |
| | mRNA | delta | AUC | delta | AUC |
| CX3CR1 | NM_001171174.1 | −1.56 | 0.84 | −0.69 | 0.67 |
| TGFBI | NM_000358.3 | −1.44 | 0.82 | −0.49 | 0.58 |
| CSF1R | NM_001349736.2 | −1.35 | 0.81 | −0.31 | 0.61 |
| HLA-DPA1 | NM_033554.3 | −1.21 | 0.88 | −0.58 | 0.68 |
| HLA-DMB | NM_002118.5 | −1.05 | 0.85 | −0.45 | 0.67 |
| HLA-DQB1 | NM_002123.5 | −0.97 | 0.81 | −0.15 | 0.55 |
| MYBL1 | NM_001080416.4 | −0.94 | 0.81 | −0.30 | 0.69 |
| HLA-DRA | NM_019111.5 | −0.92 | 0.85 | −0.50 | 0.61 |
| HLA-DRB3 | NM_022555.3 | −0.90 | 0.83 | −0.62 | 0.62 |
| CD160 | NM_007053.4 | −0.89 | 0.83 | −0.54 | 0.69 |
| HLA-DPB1 | NM_002121.6 | −0.88 | 0.85 | −0.20 | 0.60 |
| MPEG1 | NM_001039396.2 | −0.76 | 0.80 | −0.33 | 0.66 |
| GIMAP1 | NM_130759.4 | −0.75 | 0.84 | −0.70 | 0.68 |
| ARL4C | NM_001282431.2 | −0.70 | 0.80 | −0.29 | 0.68 |
| CD4 | NM_000616.5 | −0.70 | 0.83 | −0.27 | 0.64 |
| HLA-DMA | NM_006120.4 | −0.70 | 0.81 | −0.25 | 0.61 |
| IL10RA | NM_001558.4 | −0.66 | 0.81 | −0.36 | 0.64 |
| TPPP3 | NM_016140.4 | −0.64 | 0.83 | −0.05 | 0.60 |
| PGAP3 | NM_033419.5 | −0.58 | 0.82 | −0.21 | 0.67 |
| MATK | NM_139355.3 | −0.57 | 0.81 | −0.10 | 0.59 |
| DOK2 | NM_003974.4 | −0.52 | 0.81 | −0.34 | 0.65 |
| TTYH2 | NM_032646.6 | −0.50 | 0.82 | −0.10 | 0.65 |
| ULK2 | NM_014683.4 | −0.47 | 0.82 | −0.15 | 0.63 |
| ITFG2 | NM_018463.4 | −0.42 | 0.80 | −0.39 | 0.68 |
| PAFAH2 | NM_000437.4 | −0.41 | 0.80 | −0.27 | 0.66 |
| PDCD4 | NM_014456.5 | −0.40 | 0.81 | −0.40 | 0.65 |
| SIGIRR | NM_001135054.2 | −0.40 | 0.81 | −0.23 | 0.68 |
| ZNF618 | NM_001318040.2 | −0.17 | 0.82 | −0.10 | 0.65 |

RNA markers whose expression is increased in bacterial infections compared to viral infections, and whose level is unaffected by severity of infection are summarized in Table 10 (severity AUROC <0.7, BV AUROC>0.8).

TABLE 10

| | |
|---|---|
| Severity | BV |

| | RefSeq | | ROC | | ROC |
|---|---|---|---|---|---|
| | mRNA | delta | AUC | delta | AUC |
| PI3 | NM_002638.4 | 0.86 | 0.63 | 1.61 | 0.81 |

RNA markers whose expression is decreased in bacterial infections compared to viral infections, and whose level is unaffected by severity of infection are summarized in Table 11 (severity AUROC <0.7, BV AUROC>0.8).

TABLE 11

| | | | | | B-V |
|---|---|---|---|---|---|
| | | Severity | ROC | | ROC |
| | | delta | AUC | delta | AUC |
| IFI27 | NM_001130080.3 | −0.42 | 0.61 | −4.84 | 0.90 |
| IFI44L | NM_001375646.1 | −1.75 | 0.70 | −4.49 | 0.91 |
| RSAD2 | NM_080657.5 | −1.35 | 0.65 | −3.60 | 0.88 |
| IFI44 | NM_006417.5 | −1.15 | 0.62 | −3.50 | 0.88 |
| ISG15 | NM_005101.4 | −1.74 | 0.68 | −3.45 | 0.89 |
| IFIT3 | NM_001549.6 | −1.52 | 0.66 | −3.05 | 0.89 |
| EPSTI1 | NM_001002264.4 | −0.65 | 0.64 | −3.01 | 0.86 |
| HERC5 | NM_016323.4 | −1.25 | 0.66 | −2.89 | 0.87 |
| LY6E | NM_002346.3 | −1.16 | 0.68 | −2.69 | 0.90 |
| IFIT1 | NM_001548.5 | −1.16 | 0.66 | −2.65 | 0.89 |
| MX1 | NM_001144925.2 | −0.98 | 0.67 | −2.64 | 0.89 |
| IFIT2 | NM_001547.5 | −1.05 | 0.66 | −2.47 | 0.84 |
| XAF1 | NM_017523.5 | −1.13 | 0.70 | −2.25 | 0.88 |
| IFI6 | NM_022872.3 | −0.79 | 0.63 | −2.15 | 0.86 |
| EIF2AK2 | NM_002759.4 | −0.63 | 0.61 | −2.09 | 0.85 |
| OAS1 | NM_016816.4 | −0.93 | 0.69 | −1.94 | 0.89 |
| ZBP1 | NM_030776.3 | −0.79 | 0.68 | −1.93 | 0.87 |
| IFIH1 | NM_022168.4 | −0.67 | 0.62 | −1.88 | 0.84 |
| OAS3 | NM_006187.4 | −1.01 | 0.68 | −1.86 | 0.88 |
| OASL | NM_003733.4 | −0.79 | 0.65 | −1.81 | 0.86 |
| DDX60 | NM_017631.6 | −0.99 | 0.64 | −1.81 | 0.87 |
| DHX58 | NM_024119.3 | −1.11 | 0.70 | −1.77 | 0.86 |
| SPATS2L | NM_015535.3 | −1.25 | 0.68 | −1.75 | 0.88 |
| USP18 | NM_017414.4 | −1.12 | 0.66 | −1.73 | 0.87 |
| LAP3 | NM_015907.3 | −0.64 | 0.65 | −1.72 | 0.80 |
| SAMD9L | NM_152703.5 | −0.54 | 0.62 | −1.71 | 0.83 |
| MT2A | NM_005953.5 | −1.07 | 0.70 | −1.64 | 0.80 |
| OAS2 | NM_016817.3 | −1.05 | 0.70 | −1.64 | 0.90 |
| SAMD9 | NM_152703.5 | −0.46 | 0.63 | −1.57 | 0.82 |
| STAT1 | NM_001384880.1 | −0.57 | 0.62 | −1.56 | 0.81 |
| PARP12 | NM_022750.4 | −0.84 | 0.69 | −1.55 | 0.87 |
| RTP4 | NM_022147.3 | −0.97 | 0.66 | −1.54 | 0.84 |
| IFI35 | NM_005533.5 | −0.55 | 0.63 | −1.43 | 0.82 |
| CMPK2 | NM_207315.4 | −0.68 | 0.63 | −1.38 | 0.88 |
| IRF7 | NM_004031.4 | −0.77 | 0.62 | −1.38 | 0.84 |
| OTOF | NM_194248.3 | −1.12 | 0.68 | −1.33 | 0.89 |
| MX2 | NM_002463.2 | −0.46 | 0.63 | −1.28 | 0.81 |
| TRIM22 | NM_006074.5 | −0.45 | 0.60 | −1.28 | 0.81 |
| PARP14 | NM_017554.3 | −0.55 | 0.64 | −1.25 | 0.83 |
| STAT2 | NM_005419.4 | −0.65 | 0.67 | −1.22 | 0.81 |
| SERPING1 | NM_000062.3 | −0.71 | 0.62 | −1.16 | 0.84 |
| ISG20 | NM_002201.6 | −0.46 | 0.69 | −1.15 | 0.83 |
| IFIT5 | NM_012420.3 | −0.72 | 0.64 | −1.14 | 0.84 |
| MOV10 | NM_001130079.3 | −0.47 | 0.63 | −1.06 | 0.83 |
| BST2 | NM_004335.4 | −0.50 | 0.65 | −1.04 | 0.82 |
| LGALS3BP | NM_005567.4 | −0.42 | 0.66 | −1.03 | 0.85 |
| DDX58 | NM_014314.4 | −0.68 | 0.66 | −1.01 | 0.82 |
| LAMP3 | NM_014398.4 | −0.59 | 0.65 | −0.97 | 0.84 |
| HESX1 | NM_003865.3 | −0.23 | 0.64 | −0.94 | 0.82 |
| UBE2L6 | NM_004223.5 | −0.76 | 0.65 | −0.87 | 0.82 |
| GALM | NM_138801.3 | −0.50 | 0.65 | −0.84 | 0.80 |
| PHF11 | NM_001040443.3 | −0.36 | 0.63 | −0.81 | 0.81 |
| TRIM38 | NM_006355.5 | −0.29 | 0.63 | −0.80 | 0.80 |
| SP140 | NM_007237.5 | −0.38 | 0.64 | −0.79 | 0.81 |
| SLFN5 | NM_144975.4 | −0.58 | 0.69 | −0.75 | 0.82 |
| TRIM5 | NM_033034.3 | −0.21 | 0.61 | −0.65 | 0.81 |
| JUP | NM_001352773.2 | −0.40 | 0.68 | −0.54 | 0.81 |
| AXL | NM_021913.5 | −0.23 | 0.67 | −0.32 | 0.82 |

Protein combinations which show very high levels of accuracy in distinguishing between severe bacterial and severe viral are presented herein below in Table 12.

TABLE 12

AUC of severe bacterial vs. severe viral

| | | TRAIL, | | |
|---|---|---|---|---|
| | | CRP and | | TIC with |
| UniProt ID | Marker | IP10 (TIC) | marker | marker |
| P15018 | LIF | 0.93 | 0.88 | 0.97 |
| P78556 | CCL20 | 0.93 | 0.92 | 0.97 |
| Q9GZV9 | FGF-23 | 0.93 | 0.87 | 0.94 |

Protein combinations which show very high levels of accuracy in distinguishing between severe viral and non-severe viral are presented herein below in Table 13.

TABLE 13

| | | AUC of severe viral vs. non-severe viral | | |
|---|---|---|---|---|
| UniProt ID | Marker | TIC | marker | TIC with marker |
| P08727 | KRT19 | 0.96 | 0.94 | 0.97 |
| P80098 | MCP-3 | 0.96 | 0.92 | 0.97 |

Protein combinations which show very high levels of accuracy in distinguishing between severe bacterial and severe viral are presented herein below in Table 14.

TABLE 14

AUC of severe bacterial vs. severe viral

| | | | | TRAIL with |
|---|---|---|---|---|
| UniProt ID | Marker | TRAIL | marker | marker |
| P80098 | MCP-3 | 0.63 | 0.84 | 0.87 |

### REFERENCES

Arias, C. A., and B. E. Murray. 2009. “Antibiotic-Resistant Bugs in the 21st Century—a Clinical Super-Challenge.” The New England Journal of Medicine 360 (5): 439-43. doi:10.1056/NEJMp0804651.
Bogaert, D, R De Groot, and P W M Hermans. 2004. “Streptococcus Pneumoniae Colonisation: The Key to Pneumococcal Disease.” The Lancet Infectious Diseases 4 (3): 144-54. doi: 10.1016/S1473-3099(04)00938-7.
Bossuyt, Patrick M, Johannes B Reitsma, David E Bruns, Constantine A Gatsonis, Paul P Glasziou, Les M Irwig, David Moher, Drummond Rennie, Henrica C. W De Vet, and Jeroen G Lijmer. 2003. “The STARD Statement for Reporting Studies of Diagnostic Accuracy: Explanation and Elaboration.” Annals of Internal Medicine 138 (1): W1-12.
Cadieux, G., and R. Tamblyn, et al. 2007. “Predictors of Inappropriate Antibiotic Prescribing among Primary Care Physicians.” CMAJ. Canadian Medical Association Journal=Journal De l′Association Medicale Canadienne 177 (8): 877-83.
“CDC—About Antimicrobial Resistance.” 2013. Accessed January 17. www(dot)cdc(dot)gov/drugresistance/about(dot)html.
“CDC—Get Smart: Fast Facts About Antibiotic Resistance.” 2011. www(dot)cdc(dot)gov/getsmart/antibiotic-use/fast-facts(dot)html.
. 2013. Accessed January 17. www(dot)cdc(dot)gov/getsmart/antibiotic-use/fast-facts(dot)html.
Cohen, Asi, Louis Bont, Dan Engelhard, Edward Moore, David Fernandez, Racheli Kreisberg-Greenblatt, Kfir Oved, Eran Eden, and John P. Hays. 2015. “A Multifaceted ‘Omics’ Approach for Addressing the Challenge of Antimicrobial Resistance.” Future Microbiology 10 (3): 365-76. doi: 10.2217/fmb 0.14.127.
Davey, P., and E. Brown, et al. 2006. “Systematic Review of Antimicrobial Drug Prescribing in Hospitals.” Emerging Infectious Diseases 12 (2): 211-16.
Del Mar, C. 1992. “Managing Sore Throat: A Literature Review. I. Making the Diagnosis.” The Medical Journal of Australia 156 (8): 572-75.
Downey, Tom. 2006. “Analysis of a Multifactor Microarray Study Using Partek Genomics Solution.” Methods in Enzymology 411: 256-70. doi:10.1016/50076-6879(06)11013-7.
Engel, Madelon F, F P Paling, A I M Hoepelman, V van der Meer, and J J Oosterheert. 2012. “Evaluating the Evidence for the Implementation of C-Reactive Protein Measurement in Adult Patients with Suspected Lower Respiratory Tract Infection in Primary Care: A Systematic Review.” Family Practice 29 (4): 383-93. doi:10.1093/fampra/cmr119.
“European Surveillance of Antimicrobial Consumption Network (ESAC-Net).” 2014. Accessed February 26. www(dot)ecdc(dot)europa(dot)eu/en/activities/surveillance/ESAC-Net/Pages/index(dot)aspx.
Falk, Gavin, and Tom Fahey. 2009. “C-Reactive Protein and Community-Acquired Pneumonia in Ambulatory Care: Systematic Review of Diagnostic Accuracy Studies.” Family Practice 26 (1): 10-21. doi:10.1093/fampra/cmn095.
Houck, P. M., and D. W. Bratzler, et al. 2002. “Pneumonia Treatment Process and Quality.” Archives of Internal Medicine 162 (7): 843-44.
Jung, C. L., M. A. Lee, and W. S. Chung. 2010. “Clinical Evaluation of the Multiplex PCR Assay for the Detection of Bacterial Pathogens in Respiratory Specimens from Patients with Pneumonia.” Korean Journal of Clinical Microbiology 13 (1): 40. doi:10.5145/KJCM.2010.13.1.40.
Kim, K. H., J. H. Shin, and S. Y. Kim. 2009. “The Clinical Significance of Nasopharyngeal Carriages in Immunocompromised Children as Assessed.” The Korean Journal of Hematology 44 (4): 220. doi:10.5045/kjh.2009.44.4.220.
Limper, M., M. D. de Kruif, A. J. Duits, D. P.M. Brandjes, and E. C. M. van Gorp. 2010. “The Diagnostic Role of Procalcitonin and Other Biomarkers in Discriminating Infectious from Non-Infectious Fever.” Journal of Infection 60 (6): 409-16. doi:10.1016/j jinf.2010.03.016.
Linder, JA, and R S Stafford. 2001. “Antibiotic Treatment of Adults with Sore Throat by Community Primary Care Physicians: A National Survey, 1989-1999.” JAMA: The Journal of the American Medical Association 286 (10): 1181-86.
Little, P. 2005. “Delayed Prescribing of Antibiotics for Upper Respiratory Tract Infection.” BMJ (Clinical Research Ed.) 331 (7512): 301-2.
Little, P. S., and I. Williamson. 1994. “Are Antibiotics Appropriate for Sore Throats? Costs Outweigh the Benefits.” BMJ (Clinical Research Ed.) 309 (6960): 1010-11.
Oved, Kfir, Asi Cohen, Olga Boico, Roy Navon, Tom Friedman, Liat Etshtein, Or Kriger, et al. 2015. “A Novel Host-Proteome Signature for Distinguishing between Acute Bacterial and Viral Infections.” PLoS ONE 10 (3): e0120012. doi:10.1371/journal.pone.0120012.
Pulcini, C., and E. Cua, et al. 2007. “Antibiotic Misuse: A Prospective Clinical Audit in a French University Hospital.” European Journal of Clinical Microbiology & Infectious Diseases: Official Publication of the European Society of Clinical Microbiology 26 (4): 277-80.
Quenot, Jean-Pierre, Charles-Edouard Luyt, Nicolas Roche, Martin Chalumeau, Pierre-Emmanuel Charles, Yann-Eric Claessens, Sigismond Lasocki, et al. 2013. “Role of Biomarkers in the Management of Antibiotic Therapy: An Expert Panel Review II: Clinical Use of Biomarkers for Initiation or Discontinuation of Antibiotic Therapy.” Annals of Intensive Care 3 (July): 21. doi:10.1186/2110-5820-3-21.
Ramilo, Octavio, Windy Allman, Wendy Chung, Asuncion Mejias, Monica Ardura, Casey Glaser, Knut M Wittkowski, et al. 2007. “Gene Expression Patterns in Blood Leukocytes Discriminate Patients with Acute Infections.” Blood 109 (5): 2066-77. doi:10.1182/blood-2006-02-002477.
Rhedin, Samuel, Ann Lindstrand, Maria Rotzén-Östlund, Thomas Tolfvenstam, Lars Ohrmalm, Malin Ryd Rinder, Benita Zweygberg-Wirgart, et al. 2014. “Clinical Utility of PCR for Common Viruses in Acute Respiratory Illness.” Pediatrics, February, peds.2013-3042. doi: 10.1542/peds 0.2013-3042.
Scott, J. G., and D. Cohen. 2001. “Antibiotic Use in Acute Respiratory Infections and the Ways Patients Pressure Physicians for a Prescription.” The Journal of Family Practice 50 (10): 853-58.
Shin, J. H., H. Y. Han, and S. Y. Kim. 2009. “Detection of Nasopharyngeal Carriages in Children by Multiplex Reverse Transcriptase-Polymerase Chain Reaction.” Korean Journal of Pediatrics 52 (12): 1358. doi:10.3345/kjp.2009.52.12.1358.
Spiro, D. M., and K. Y. Tay, et al. 2006. “Wait-and-See Prescription for the Treatment of Acute Otitis Media: A Randomized Controlled Trial.” JAMA: The Journal of the American Medical Association 296 (10): 1235-41.
Spuesens, Emiel B. M., Pieter L. A. Fraaij, Eline G. Visser, Theo Hoogenboezem, Wim C. J. Hop, Léon N. A. van Adrichem, Frank Weber, et al. 2013. “Carriage of Mycoplasma Pneumoniae in the Upper Respiratory Tract of Symptomatic and Asymptomatic Children: An Observational Study.” PLoS Med 10 (5): e1001444. doi:10.1371/journal.pmed.1001444.
Tang, Benjamin MP, Guy D Eslick, Jonathan C Craig, and Anthony S McLean. 2007. “Accuracy of Procalcitonin for Sepsis Diagnosis in Critically Ill Patients: Systematic Review and Meta-Analysis.” The Lancet Infectious Diseases 7 (3): 210-17. doi:10.1016/S1473-3099(07)70052-X.
“Threat Report 2013|Antimicrobial Resistance|CDC.” 2013. Accessed November 10. www(dot)cdc(dot)gov/drugresistance/threat-report-2013/.
Tian, Qiang, Serguei B. Stepaniants, Mao Mao, Lee Weng, Megan C. Feetham, Michelle J. Doyle, Eugene C. Yi, et al. 2004. “Integrated Genomic and Proteomic Analyses of Gene Expression in Mammalian Cells.” Molecular & Cellular Proteomics: MCP 3 (10): 960-69. doi:10.1074/mcp.M400055-MCP200.
Uyeki, Timothy M, Ramakrishna Prasad, Charles Vukotich, Samuel Stebbins, Charles R Rinaldo, Yu-Hui Ferng, Stephen S Morse, et al. 2009. “Low Sensitivity of Rapid Diagnostic Test for Influenza.” Clinical Infectious Diseases: An Official Publication of the Infectious Diseases Society of America 48 (9): e89-92. doi:10.1086/597828.
van der Meer, Victor, Arie Knuistingh Neven, Peterhans J van den Broek, and Willem J J Assendelft. 2005. “Diagnostic Value of C Reactive Protein in Infections of the Lower Respiratory Tract: Systematic Review.” BMJ (Clinical Research Ed.) 331 (7507): 26. doi:10.1136/bmj.38483.478183.EB.
“WHO|Antimicrobial Resistance.” 2013. Accessed December 5. www(dot)who(dot)int/mediacentre/factsheets/fs194/en/index(dot)html.
“WHO Europe-Data and Statistics.” 2014. Accessed February 24. www(dot)euro(dot)who(dot)int/en/health-topic s/disease-prevention/antimicrobial-resistance/data-and-statistics.

1. A method of treating a subject having an infectious disease comprising:
(a) measuring the amount of at least one protein set forth in Table 3 and/or Table 4 and at least one protein set forth in Table 6 in a sample derived from the subject;
(b) ruling in or ruling out a viral disease based on said amount of said at least one protein set forth in Table 4 and/or ruling in or ruling out a bacterial disease based on said amount of said at least one protein set forth in Table 3;
(c) determining the severity of the viral disease and/or bacterial disease based on said amount of said at least one protein set forth in Table 6, thereby classifying the infectious disease; and
(d) treating the subject according to the classification of the infection.

2-3. (canceled)

4. The method of claim 1, wherein the classifying is not based on the amount of a protein that is differentially expressed in both (a) bacterial and viral infections; and (b) in severe and non-severe infections.

5. The method of claim 1, wherein said at least one protein set forth in Table 4 is selected from the group consisting of LAMP3, LAGS, CXCL11 and MCP-2.

6. The method of claim 1, wherein said at least one protein set forth in Table 3 is OSM or CCL25.

7. The method of claim 1, wherein said at least one protein set forth in Table 6 is selected from the group consisting of FGF-23, IL10, CCL20, IL8, STC1, HNMT, AREG, OPG, DCBLD2, PRDX1, PSIP1 and HEXIM1.

8-11. (canceled)

12. A method of treating a subject showing signs of a severe infection comprising:
(a) measuring the amounts of TRAIL, CRP, IP10 and at least one additional protein selected from the group consisting of LIF, CCL20 and FGF-23;
(b) ruling in a bacterial or viral disease based on said amounts; and
(c) treating the infection based on said ruling.

13. A method of treating a subject having a viral disease comprising:
(a) measuring the amounts of TRAIL, CRP, IP10 and at least one additional protein selected from the group consisting of KRT19 and MCP-3;
(b) determining the severity of the viral disease based on said amounts; and
(c) treating the subject based on step (b).

14-16. (canceled)

17. The method of claim 1, wherein the subject shows symptoms of an infectious disease.

18. (canceled)

19. The method of claim 1, wherein the subject does not have a chronic non-infectious disease.

20. The method of claim 1, wherein the sample is whole blood or a fraction thereof.

21-22. (canceled)

23. The method of claim 1, wherein the level of no more than 10 proteins is used to classify the infection.

24. The method of claim 1, wherein no more than 5 proteins are measured to determine the infection type.

25-42. (canceled)
